SEQUENCE LISTING (1 sequence)

SEQ ID NO: 1 - DNA, 8 bp, Homo sapiens
tacgtgct

(19) United States
(12) Patent Application Publication
(10) Pub. No.: US 2005/0148496 A1
(43) Pub. Date: Jul. 7, 2005

(54) Treatment of rheumatoid arthritis with hypoxia inducible factor-1alpha antagonists

(75) Inventors: Nadine Defranoux, San Francisco, CA (US); Vincent Jacques Hurez, Albany, CA (US); Seth G. Michelson, San Jose, CA (US); Lisl Katharine Shoda, Menlo Park, CA (US); Leif Gustaf Wennerberg, Mountain View, CA (US)

Correspondence Address: FISH & RICHARDSON P.C., PO BOX 1022, MINNEAPOLIS, MN, 55440-1022, US

(73) Assignee: Entelos, Inc., Foster City, CA (US)

(21) Appl. No.: 10/997,764

(22) Filed: Nov. 24, 2004

Related U.S. Application Data
(60) Provisional application No. 60/525,363, filed on Nov. 26, 2003.

(51) Int. Cl. A61K 38/17; A61K 48/00

(52) U.S. Cl. 514/2; 514/44

(57) ABSTRACT

The invention encompasses a novel method of treating inflammatory disease, such as rheumatoid arthritis, and novel methods of identifying and screening for drugs useful in the treatment of inflammatory diseases and their clinical symptoms. The inventors have made the discovery that the activity of HIF-1α, a transcription regulator known to have an effect on some cancers, has a significant impact on the pathophysiology of rheumatoid arthritis. The symptoms of an inflammatory disease, such as rheumatoid arthritis, may be alleviated by administering a compound that inhibits the activity of HIF-1α.

### A. RELATED APPLICATIONS

This application claims the benefit of U.S. Provisional Application No. 60/525,363, filed Nov. 26, 2003, which is herein incorporated by reference.

### B. FIELD OF THE INVENTION

This invention relates to novel methods of treating rheumatoid arthritis and methods of identifying compounds useful in treating rheumatoid arthritis.

### C. BACKGROUND OF THE INVENTION

There are more than 100 forms of arthritis and of them, rheumatoid arthritis is the most painful and crippling form. Rheumatoid arthritis, a common disease of the joints, is an autoimmune disease that affects over 2 million Americans, with a significantly higher occurrence among women than men. In rheumatoid arthritis, the membranes or tissues (synovial membranes) lining the joints become inflamed (synovitis). Over time, the inflammation may destroy the joint tissues, leading to disability. Because rheumatoid arthritis can affect multiple organs of the body, rheumatoid arthritis is referred to as a systemic illness and is sometimes called rheumatoid disease. The onset of rheumatoid disease is usually in middle age, but frequently occurs in one's 20s and 30s. See the Merck Manual, Sixteenth Edition, section 106 for a further discussion.

The pain and whole-body (systemic) symptoms associated with rheumatoid disease can be disabling. Over time, rheumatoid arthritis can cause significant joint destruction, leading to deformity and difficulty with daily activities. It is not uncommon for people with rheumatoid arthritis to suffer from some degree of depression, which may be caused by pain and progressive disability. A study reports that one-fourth of people with rheumatoid arthritis are unable to work by 6 to 7 years after their diagnosis, and half are not able to work after 20 years (O'Dell J R (2001). Rheumatoid arthritis: The clinical picture. In W J Koopman, ed., Arthritis and Allied Conditions: A Textbook of Rheumatology, 14th ed., vol. 1, chap. 58, pp. 1153-1186. Philadelphia: Lippincott Williams and Wilkins). Musculoskeletal conditions such as rheumatoid arthritis cost the U.S. economy nearly $65 billion per year in medical care and indirect expenses such as lost wages and production.

Synovial inflammation, rapid degradation of cartilage, and erosion of bone in affected joints are characteristic of rheumatoid arthritis (RA). Recent evidence indicates that skeletal tissue degradation and inflammation are regulated through overlapping but not identical biological processes in the rheumatoid joint and that therapeutic effects on these two aspects need not be correlated. Due to the complexity of the biological processes in the joint, mathematical and computer models can be used to help better understand the interactions between the various tissue compartments, cell types, mediators, and other factors involved in joint disease and healthy homeostasis. Several researchers have constructed simple models of the mechanical environment of the joint, rather than the biological processes of rheumatoid arthritis, and compared the results to patterns of disease and development in cartilage and bone (Wynarsky & Greenwald, J. Biomech., 16: 241-251, 1983; Pollatschek & Nahir, J. Theor. Biol., 143: 497-505, 1990; Beaupre et al., J. Rehabil. Res. Dev., 37: 145-151, 2000; Shi et al., Acta Med. Okayama, 17: 646-653, 1999). A computer manipulable mathematical model of joint diseases that includes multiple compartments including the synovial membrane and the interactions of these compartments is described in published PCT application WO 02/097706, published 5 Dec. 2002 and U.S. patent application Ser. No. 10/154,123, published 24 Apr. 2003 as 2003-0078759. Both publications are incorporated herein by reference in their entirety.

Rheumatoid arthritis is a chronic disease that, at present, can be controlled but not cured. The goal of treatment is relief of symptoms and keeping the disease from getting worse. The goals of most treatments for rheumatoid arthritis are to relieve pain, reduce inflammation, slow or stop the progression of joint damage, and improve a person's ability to function. Current approaches to treatment include lifestyle changes, medication, surgery, and routine monitoring and care. Medications used for the treatment of rheumatoid arthritis can be divided into two groups based on how they affect the progression of the disease: (1) symptom-relieving drugs and (2) disease-modifying drugs.

Medications to relieve symptoms, such as pain, stiffness, and swelling, may be used. Nonsteroidal anti-inflammatory drugs (NSAIDs), such as aspirin, ibuprofen, and naproxen are used to control pain and may help reduce inflammation. They do not control the disease or stop the disease from getting worse. Corticosteroids, such as prednisone and methylprednisolone (Medrol), are used to control pain and reduce inflammation. They may control the disease or stop the disease from getting worse; however, using corticosteroids as the only therapy for an extended time is not considered the best treatment. Corticosteroids are often used to control symptoms and flares of joint inflammation until anti-rheumatic drugs reach their full effectiveness, which can take up to 6 months. Nonprescription medications such as acetaminophen and topical medications such as capsaicin are used to control pain, but do not usually affect joint swelling or worsening of the disease.

Disease-modifying anti-rheumatic drugs (DMARDs) are used to control the progression of rheumatoid arthritis and to try to prevent joint deterioration and disability. These anti-rheumatic drugs are often given in combination with other anti-rheumatic drugs or with other medications, such as nonsteroidal anti-inflammatory drugs. Disease-modifying anti-rheumatic drugs commonly prescribed for rheumatoid arthritis include antimalarial medications such as hydroxycholoroquine (Plaquenil) or chloroquine (Aralen), methotrexate (e.g., Rheumatrex), sulfasalazine (Azulfidine), leflunomide (Arava), etanercept (Enbrel), infliximab (Remicade), adalimumab (Humira) and anakinra (Kineret). DMARDs less commonly prescribed for rheumatoid arthritis include azathioprine (Imuran), penicillamine (e.g., Cuprimine or Depen), gold salts (e.g., Ridaura or Aurolate), minocycline (e.g., Dynacin or Minocin), cyclosporine (e.g., Neoral or Sandimmune), and cyclophosphamide (e.g., Cytoxan or Neosar). Some of these anti-rheumatic drugs can take up to 6 months to work. Many have serious side effects.

Thus a need exists for new, therapeutically effective drugs for the treatment of rheumatoid arthritis as well as new methods for identifying such drugs.

### D. SUMMARY OF THE INVENTION

In one aspect, the invention provides methods for alleviating at least one symptom of rheumatoid arthritis comprising administering a therapeutically effective amount of an antagonist of Hypoxia-Inducible Factor 1α (HIF-1α) activity to a patient having rheumatoid arthritis. In a preferred embodiment, the antagonist decreases the HIF-1α activity by at least 45%, more preferably by at least 85% and most preferably by at least 95%. The antagonist of HIF-1α activity may be a protein, nucleic acid or small molecule inhibitor. A “small molecule” is defined herein as a molecule having a molecular weight of less than 1000 daltons. Preferred antagonists include, but are not limited to, YC-1 stimulator (sGC), 2-methoxyestradiol, taxol, vincristine, 1-methylpropyl-2-imidazolyl disulphide, pleurotin, rapamycin/CC1779, LY294002, wortmannin, geldanamycin, camptothecin (and analogs, such as Topotecan), PD98059, quinocarmycin (and analogs such as NCS-607097 (DX-52-1)), and phosphate and tension homologue (PTEN). In preferred embodiments, the patient is a methotrexate resistant patient, a TNF-α blockade cartilage nonresponder (CNR), a TNF-α blockade hyperplasia nonresponder (HNR), or a TNF-α blockade double nonresponder (DNR).

In another aspect, the invention provides methods for decreasing density of synovial cells in a joint comprising administering a therapeutically effective amount of an antagonist of HIF-1α activity to a patient having a condition associated with abnormally increased synovial cell density. In a preferred embodiment, the antagonist decreases the HIF-1α activity by at least 45%, more preferably by at least 85% and most preferably by at least 95%.

The invention also provides methods for decreasing cartilage degradation in a joint comprising administering a therapeutically effective amount of an antagonist of HIF-1α activity to a patient having a condition associated with an abnormally high rate of cartilage degradation. In a preferred embodiment, the antagonist decreases the HIF-1α activity by at least 45%, more preferably by at least 85% and most preferably by at least 95%.

Yet another aspect of the invention provides methods for decreasing IL-6 concentration in synovial tissue comprising administering a therapeutically effective amount of an antagonist of HIF-1α activity to a patient having a condition associated with an abnormally high concentration of IL-6 in synovial tissue. In a preferred embodiment, the antagonist decreases the HIF-1α activity by at least 45%, more preferably by at least 85% and most preferably by at least 95%.

In another aspect, the invention provides methods of alleviating at least one symptom of an inflammatory disease comprising administering a therapeutically effective amount of an antagonist of HIF-1α activity to a patient suffering from an inflammatory disease. In a preferred embodiment, the antagonist decreases the HIF-1α activity by at least 45%, more preferably by at least 85% and most preferably by at least 95%. In preferred embodiments, the inflammatory disease is selected from the group consisting of diabetes, arteriosclerosis, inflammatory aortic aneurysm, restenosis, ischemia/reperfusion injury, glomerulonephritis, reperfusion injury, rheumatic fever, systemic lupus erythematosus, rheumatoid arthritis, Reiter's syndrome, psoriatic arthritis, ankylosing spondylitis, coxarthritis, inflammatory bowel disease, ulcerative colitis, Crohn's disease, pelvic inflammatory disease, multiple sclerosis, osteomyelitis, adhesive capsulitis, oligoarthritis, osteoarthritis, periarthritis, polyarthritis, psoriasis, Still's disease, synovitis, Alzheimer's disease, Parkinson's disease, amyotrophic lateral sclerosis, osteoporosis, and inflammatory dermatosis. More preferably the inflammatory disease is an arthritis, such as rheumatoid arthritis, psoratic arthritis, coxarthritis, osteoarthritis, or polyarthritis. Most preferably, the inflammatory disease is rheumatoid arthritis.

Yet another aspect of the invention provides methods of alleviating at least one symptom of rheumatoid arthritis, comprising administering an antagonist of HIF-1α activity and an anti-rheumatic drug to a patient having rheumatoid arthritis. The anti-rheumatic drug can be any drug that, in combination with HIF-1α antagonism, provides a better clinical outcome than treatment with HIF-1α antagonism or the anti-rheumatic drug alone. The anti-rheumatic drug can be a symptom-relieving anti-rheumatic drug or a disease-modifying anti-rheumatic drug. Exemplary symptom-relieving anti-rheumatic drugs include aspirin, ibuprofen, naproxen, and corticosteroids, such as prednisone and methylprednisolone (Medrol). Exemplary disease-modifying anti-rheumatic drugs include hydroxycholoroquine (Plaquenil), chloroquine (Aralen), methotrexate (e.g., Rheumatrex), sulfasalazine (Azulfidine), leflunomide (Arava), etanercept (Enbrel), infliximab (Remicade), adalimumab (Humira), anakinra (Kineret), azathioprine (Imuran), penicillamine (e.g., Cuprimine or Depen), gold salts (e.g., Ridaura or Aurolate), minocycline (e.g., Dynacin or Minocin), cyclosporine (e.g., Neoral or Sandimmune), and cyclophosphamide (e.g., Cytoxan or Neosar). In preferred embodiments, the anti-rheumatic drug is methotrexate, a TNF-α antagonist, an interleukin-1 receptor antagonist, such as Anakinra, or a steroid, such as methylprednisolone.

Another aspect of the invention provides methods for manufacturing a drug for use in the treatment an inflammatory disease comprising: (a) identifying a compound as useful in the treatment of inflammatory disease and formulating said compound for human consumption. The compound is identified by (i) comparing an amount of HIF-1α activity in the presence of the compound with an amount HIF-1α activity in the absence of the compound; and (ii) identifying the compound as useful in the treatment of rheumatoid arthritis when the amount of HIF-1α activity in the presence of the compound is lower than the amount of HIF-1α activity in the absence of the compound. Preferably, the inflammatory disease is rheumatoid arthritis. Preferably, the amount of HIF-1α activity in the presence of the compound is at least 45% lower than the amount of HIF-1α activity in the absence of the compound. More preferably, the compound will decrease the activity of HIF-1α by at least 85%. Most preferably, the amount of HIF-1α activity in the presence of the compound is at least 95% lower than the amount of HIF-1α activity in the absence of the compound.

Another aspect of the invention provides methods for identifying a compound useful in the treatment of inflammatory disease, which method comprises: (a) comparing an amount of HIF-1α activity in the presence of the compound with an amount HIF-1α activity in the absence of the compound; and (b) selecting the compound as useful in the treatment of inflammatory disease when the amount of HIF-1α activity in the presence of the compound is lower than the amount of HIF-1α activity in the absence of the compound. Preferably, the inflammatory disease is rheumatoid arthritis. In one embodiment, a collection of compounds may be screened by repeating steps (a) and (b) for each compound in a collection of compounds, wherein at least one compound of the collection is selected as useful for the treatment of an inflammatory disease, e.g., rheumatoid arthritis.

The amount of HIF-1α activity can be determined by a variety of methods. One method of the invention comprises measuring an amount of HIF-1α bound to a hypoxia-responsive element (HRE) or measuring an amount of transcription from an HIF-1α-responsive gene. Preferred HIF-1α-responsive genes include VEGF, Glut-1, enolase 1, and aldolase A.

An alternative method for measuring HIF-1α activity comprises comparing an amount of leukocytes that migrate through at least one layer of endothelial cells in the presence of the compound with an amount of leukocytes that migrate through at least one layer of endothelial cells in the absence of the compound. Preferably, the leukocytes are T-cells or monocytes and most preferably are monocytes. In a preferred embodiment of the invention, the compound is identified or selected as useful in the treatment of an inflammatory disease, preferably rheumatoid arthritis, when the amount of leukocytes that migrate in the presence of the compound is at least 30% lower than the amount of leukocytes that migrate in the absence of the compound. More preferably, the compound will decrease leukocyte migration by at least 40% and most preferably by at least 50%.

Yet another method for measuring a decrease in HIF-1α activity comprises observing an amount of leukocyte apoptosis in the presence of the compound that is higher than an amount of leukocyte apoptosis in the absence of the compound. Preferably, the compound useful for the treatment of an inflammatory disease, preferably rheumatoid arthritis, will cause an increase in macrophage apoptosis. In a preferred embodiment of the invention, the compound is identified or selected as useful in the treatment of rheumatoid arthritis when the amount of macrophage apoptosis in the presence of the compound is at least 1.25-fold greater than the amount of macrophage apoptosis in the absence of the compound. More preferably the compound will increase macrophage apoptosis by at least 1.5-fold and most preferably by at least 1.7-fold.

The amount of macrophage apoptosis may be determined by any apoptosis measurement technique, now known or discovered in the future. One embodiment of the invention measures the amount of macrophage apoptosis by a process comprising the steps of exposing a population of cells to an inducer of apoptosis in the presence or absence of the compound, and measuring the percentage of cells having DNA fragmentation, wherein the percentage of cells having DNA fragmentation represents the amount of macrophage apoptosis. The percentage of cells having DNA fragmentation may be measured by any method know in the art, including propidium iodide uptake or TUNEL (terminal deoxynucleotidyl transferase-mediated 2′-deoxyuridine 5′-triphosphate-biotin nick-end labeling) assay. In yet another embodiment of the invention, the amount of macrophage apoptosis is measured by a process comprising the steps of exposing a population of cells to an inducer of apoptosis in the presence or absence of the compound, and measuring the percentage of cells expressing phosphatidylserine on the extracellular surface of the cell membrane, wherein the percentage of cells expressing phosphatidylserine on the extracellular surface of the cell membrane represents the amount of macrophage apoptosis. Preferably the expression of phosphatidylserine on the extracellular surface of the cytoplasmic membrane is measured by binding of annexin V to the phosphatidylserine. Preferred enducers of apoptosis include, but are not limited to, sFas ligand, anti-Fas or TRAIL or hypoxia.

In yet another aspect of the invention, measuring the amount of HIF-1α activity comprises comparing an amount of angiogenesis in the presence of the compound with an amount of angiogenesis in the absence of the compound, wherein the amount of angiogenesis represents the amount HIF-1α activity. Preferably, the compound is identified or selected as useful in the treatment of an inflammatory disease, such as rheumatoid arthritis, when the amount of angiogenesis in the presence of the compound is at least 25% lower than the amount of angiogenesis in the absence of the compound. More preferably the compound will decrease the amount of angiogenesis by at least 35% and most preferably by at least 50%. The amount of angiogenesis may be determined by any angiogenesis measurement technique, now known or discovered in the future. In a preferred embodiment of the invention, the amount of angiogenesis is measured by (1) providing a layer of basement proteins, (2) culturing a population of endothelial cells on the layer of basement proteins in the presence or absence of the compound, and (3) quantifying the number of capillaries formed. Capillary formation may be quantified by visualizing cell tubes, counting branch points or calculating the total capillary length in a view field.

It will be appreciated by one of skill in the art that the embodiments summarized above may be used together in any suitable combination to generate additional embodiments not expressly recited above, and that such embodiments are considered to be part of the present invention

### II. BRIEF DESCRIPTION OF THE FIGURES

For a better understanding of the nature and objects of some embodiments of the invention, reference should be made to the following detailed description taken in conjunction with the accompanying drawings, in which:

FIG. 1 demonstrates the effect of HIF-1α blockade on synovial cell density in a typical rheumatoid arthritis patient.

FIG. 2 demonstrates the effect of HIF-1α blockade on cartilage degradation in a typical rheumatoid arthritis patient.

FIG. 3 demonstrates the effect of HIF-1α blockade on IL-6 in synovial tissue in a typical rheumatoid arthritis patient.

FIG. 4 demonstrates simulation of HIF-1α blockade on individual significant biological processes in a typical rheumatoid arthritis patient.

FIG. 5 demonstrates simulation of HIF-1α blockade on individual significant biological processes in a methotrexate resistant patient.

FIG. 6 demonstrates the effect of HIF-1α blockade on synovial cell density in a methotrexate resistant patient.

FIG. 7 demonstrates the effect of HIF-1α blockade on cartilage degradation in a methotrexate resistant patient.

FIG. 8 demonstrates the effect of HIF-1α blockade on IL-6 in synovial tissue in a methotrexate resistant patient.

FIG. 9 provides a comparison of HIF-1α inhibition with expected increase of macrophage apoptosis and decreased monocyte recruitment rates at the ‘upper maximum effect’ of HIF-1α antagonism in a typical rheumatoid arthritis patient.

FIG. 10 provides a comparison of HIF-1α inhibition with expected increase of macrophage apoptosis and decreased monocyte recruitment rates at the ‘most likely maximum effect’ of HIF-1α antagonism in a typical rheumatoid arthritis patient.

FIG. 11 illustrates the factors that control, or are controlled by, hypoxia-inducible factor-1α (HIF-1α) in inflammatory sites with low oxygen levels. Upper left box: hypoxia inhibits prolyl hydroxylase enzymes, which degrades HIF-1α. Upper right box: HIF-1α is needed for several aspects of inflammation, namely the redness and swelling of injured tissues and, via glycolytic enzymes, leukocyte migration into injured areas; HIF-1α also induces the production of vascular endothelial growth factor (VEGF). Lower box: HIF-1α increases the production of nitric oxide (NO).

### III. DETAILED DESCRIPTION

A. Overview

In general this invention can be viewed as encompassing a novel method of treating inflammatory disease, such as rheumatoid arthritis, and novel methods of identifying and screening for drugs useful in the treatment of inflammatory diseases and their clinical symptoms. The inventors have made the discovery that the activity of HIF-1α, a transcription regulator known to have an effect on some cancers, has a significant impact on the pathophysiology of rheumatoid arthritis. The symptoms of an inflammatory disease, such as rheumatoid arthritis, may be alleviated by administering a compound that inhibits the activity of HIF-1α.

B. Definitions

The term “abnormally increased synovial cell density,” as used herein, refers to a condition in which the synovial tissue of a joint contains a number of synovial cells that is at least ten-times higher than the number of synovial cells found in the synovial tissue of a normal, i.e., non-diseased, joint.

The term “abnormally high rate of cartilage degradation,” as used herein, refers to a detectable joint space narrowing as determined by standard radiographic measures. In a non-diseased joint narrowing is not detectable.

The term “abnormally high concentration of IL-6 in synovial tissue,” as used herein, refers to a level of IL-6 in the synovial tissue of the diseased joint that is at least 3 standard deviations higher than that found in a normal, non-diseased, joint.

“Administering” means any of the standard methods of administering a pharmaceutical composition known to those skilled in the art. Examples include, but are not limited to intravenous, intramuscular or intraperitoneal administration.

The term “antagonist of HIF-1α activity,” as used herein, refers to the property of inhibiting any one of the three biological activities of HIF-1α shown to be relevant to rheumatoid arthritis: (1) monocyte/macrophage and T-cell recruitment, (2) macrophage and T-cell apoptosis, and (3) macrophage cytokine production. Inhibition need not be 100% effective in order to be antagonistic. In addition, inhibition of HIF-1α activity may be achieved by decreasing expression of HIF-1α, by increasing ubiquitylation and proteolysis of HIF-1α or by interfering with transcription activation of HIF-1-responsive genes.

The term “drug” refers to a compound of any degree of complexity that can affect a biological system, whether by known or unknown biological mechanisms, and whether or not used therapeutically. Examples of drugs include typical small molecules (a molecule having a molecular weight of less than 1000 daltons) of research or therapeutic interest; naturally-occurring factors such as endocrine, paracrine, or autocrine factors, antibodies, or factors interacting with cell receptors of any type; intracellular factors such as elements of intracellular signaling pathways; factors isolated from other natural sources; pesticides; herbicides; and insecticides. Drugs can also include, agents used in gene therapy such as DNA and RNA. Also, antibodies, viruses, bacteria, and bioactive agents produced by bacteria and viruses (e.g., toxins) can be considered as drugs. A response to a drug can be a consequence of, for example, drug-mediated changes in the rate of transcription or degradation of one or more species of RNA, drug-mediated changes in the rate or extent of translational or post-translational processing of one or more polypeptides, drug-mediated changes in the rate or extent of degradation of one or more proteins, drug-mediated inhibition or stimulation of action or activity of one or more proteins, and so forth. In some instances, drugs can exert their effects by interacting with a protein. For certain applications, drugs can also include, for example, compositions including more than one drug or compositions including one or more drugs and one or more excipients.

“Inflammatory diseases” refers to a class of diverse diseases and disorders that are characterized by any one of the following: the triggering of an inflammatory response; an upregulation of any member of the inflammatory cascade; the downregulation of any member of the inflammatory cascade. Inflammatory diseases include diabetes, arteriosclerosis, inflammatory aortic aneurysm, restenosis, ischemia/reperfusion injury, glomerulonephritis, reperfusion injury, rheumatic fever, systemic lupus erythematosus, rheumatoid arthritis, Reiter's syndrome, psoriatic arthritis, ankylosing spondylitis, coxarthritis, inflammatory bowel disease, ulcerative colitis, Crohn's disease, pelvic inflammatory disease, multiple sclerosis, osteomyelitis, adhesive capsulitis, oligoarthritis, osteoarthritis, periarthritis, polyarthritis, psoriasis, Still's disease, synovitis, Alzheimer's disease, Parkinson's disease, amyotrophic lateral sclerosis, osteoporosis, and inflammatory dermatosis. The singular term “inflammatory disease” includes any one or more diseases selected from the class of inflammatory diseases, and includes any compound or complex disease state wherein a component of the disease state includes a disease selected from the class of inflammatory diseases.

The term “joint,” as used herein, comprises the synovial tissue, synovial fluid, articular cartilage, bone tissues, and their cellular and extracellular composition, and the soluble mediators they contain.

The term “methotrexate resistant patient” refers to a rheumatoid arthritis patient who does not effectively respond to methotrexate treatment or who initially responds to methotrexate and becomes refractory over time.

As used herein, “normoxic conditions” refers to physiologic levels of tissue oxygenation (PO2 varying from 40 to 150 mmHg depending on the tissue). The phrase, “hypoxic conditions” refers to levels of tissue oxygenation lower than physiologic levels, i.e., below 40 mmHg.

The term “patient” refers to any warm-blooded animal, preferably a human. Patients having rheumatoid arthritis can include, for example, patients that have been diagnosed with rheumatoid arthritis, patients that exhibit one or more of the symptoms associated with rheumatoid arthritis, or patients that are progressing towards or are at risk of developing rheumatoid arthritis.

As used herein, a “therapeutically effective amount” of a drug of the present invention is intended to mean that amount of the compound that will inhibit an increase in synovial cells in a rheumatic joint or decrease the rate of cartilage degradation in a rheumatic joint or decrease IL-6 concentration in synovial tissue, and thereby cause the regression and palliation of the pain and inflammation associated with rheumatoid arthritis.

The term “TNF-α blockade resistant patient” refers to a rheumatoid arthritis patient who does not effective respond to TNF-α blockade or who initially responds to TNF-α blockade and becomes refractory over time.

The term “TNF-α blockade cartilage nonresponder” refers to a rheumatoid arthritis patient with low initial TNF-α activity who shows decreased synovial hyperplasia, but minimal reduction in cartilage degradation in response to TNF-α blockade.

The term “TNF-α blockade hyperplasia nonresponder” refers to a rheumatoid arthritis patient with abnormally high or resistant levels of TNF-α activity who yields improvement in cartilage degradation but little decrease in synovial hyperplasia in response to TNF-α blockade.

The term “TNF-α blockade double nonresponder” refers to a rheumatoid arthritis patient with negligible initial TNF-α activity who shows poor response in both synovial hyperplasia and cartilage degradation in response to TNF-α blockade.

C. Identifying a Compound Useful in Treating an Inflammatory Disease

One aspect of the invention is a method of identifying a compound useful in the treatment of an inflammatory disease, which method comprises (a) comparing an amount of HIF-1α activity in the presence of the compound with an amount HIF-1α activity in the absence of the compound; and (b) selecting the compound as useful in the treatment of inflammatory disease when the amount of HIF-1α activity in the presence of the compound is lower than the amount of HIF-1α activity in the absence of the compound. Preferably, the inflammatory disease is rheumatoid arthritis. The dynamic processes related to the biological state of a human joint afflicted with rheumatoid arthritis involve various biological variables related to the processes involved in cartilage metabolism, tissue inflammation, and tissue hyperplasia, including the following:
macrophage population dynamics including recruitment, activation, proliferation, apoptosis and their regulation,
T cell population dynamics including recruitment, antigen-dependent and antigen-independent activation, proliferation, apoptosis and their regulation
Fibroblast-like synoviocyte (FLS) population dynamic including influx in the tissue, proliferation, and apoptosis and their regulation
chondrocyte population dynamics including: proliferation and apoptosis
synthesis and regulation of a variety of proteins, including growth factors, cytokines, chemokines, proteolytic enzymes and matrix proteins, by the different cell type represented (macrophages, FLS, T cells, chondrocytes).
expression of adhesion molecules by endothelial cells
transport of mediators between synovial tissue and cartilage
interaction between cytokines or proteases and their natural inhibitors, antigen presentation, and
binding of therapeutic agents to cellular mediators (TNF-α antagonists, such as etanercept and infliximab, and IL-IR antagonists, such as anakinra).
   Based on observations of an in silico model providing mathematical representations of a human joint afflicted with rheumatoid arthritis, we found that antagonists of HIF-1α will alleviate the symptoms of rheumatoid arthritis, especially decreasing the density of synovial cells, decreasing cartilage degradation, decreasing bone erosion and decreasing IL-6 concentration in synovial tissue. These observations also take into account vascular volume and the effect of therapeutic agents such as methotrexate, steroids, non-steroidal anti-inflammatory drugs, soluble TNF-α receptor, TNF-α antibody, and interleukin-1 receptor antagonists.

In silico modeling integrates relevant biological data—genomic, proteomic, and physiological—into a computer-based platform to reproduce a system's control principles. Given a set of initial conditions representing a defined disease state, these computer-based models can simulate the system's future biological behavior, a process termed biosimulation. The present invention arose from observations of these conditions.

Using a “top-down” approach that starts by defining a general set of behaviors indicative of rheumatoid arthritis, these behaviors are used as constraints on the system. A set of nested subsystems is developed to define the next level of underlying detail. For example, given a behavior such as cartilage degradation in rheumatoid arthritis, the specific mechanisms inducing that behavior are each modeled in turn, yielding a set of subsystems, which themselves are deconstructed and modeled in detail. The control and context of these subsystems is, therefore, already defined by the behaviors that characterize the dynamics of the system as a whole. The deconstruction process continues modeling more and more biology, from the top down, until there is enough detail to replicate the known biological behavior of rheumatoid arthritis.

When using a top-down approach, data is identified and collected to support two specific purposes: (1) describing basic biology and (2) describing physiological function or behavior of the whole system. Data describing physiological functions or behavior of the whole system are selected early in the development of the model. These data represent the broad range of behaviors of the models system, i.e. cartilage degradation as a measurement (behavior) of rheumatoid arthritis patients. These data are human in vivo data based on well-established clinical trials. Data describing basic biology is selected to sufficiently model the subsystems required to simulate the selected behaviors. These data can be human or animal (where human is preferred but not always available) in vivo, in vitro, or ex vivo data which provide an understanding of the underlying biology.

The top-down approach was used to develop a model of rheumatoid arthritis in a human joint. A similar model is described in co-pending U.S. patent application Ser. No. 10/154,123, published 24 Apr. 2003 as 2003-0078759. Three key clinical outcomes are of particular interest in the present model: synovial cell density, the rate of cartilage degradation, and the level of IL-6 in synovial tissue. Rheumatoid arthritis is a systemic inflammatory disease with elevated levels of proinflammatory cytokines in peripheral blood, especially IL-6. C-reactive protein (CRP) is a common marker of inflammation which is routinely measured in the plasma, and several studies have shown a correlation between the concentration of IL-6 and the concentration of CRP in rheumatoid arthritis patients. Therefore, IL-6 concentration in either the joint or the plasma represents a good marker of inflammation.

The explicit representation of the underlying biology of the disease allows the modulation of each subsystem alone or in combination to identify the one(s) with most impact on a specific clinical outcome, such as cartilage degradation or synovial cell density. By focusing modeling and data collection efforts on those subsystems with the greatest impact on the phenotypic onset and progression or rheumatoid arthritis, this approach can help more clearly represent the system's complexity and identify causal factors underlying the pathophysiology of rheumatoid arthritis. By modulating, in silico, each subsystem (e.g. knocking-out one cell type or intercellular mediator, or blocking one particular biological process), its contribution to the overall disease pathophysiology can be evaluated to better understand the biological phenomena driving rheumatoid arthritis, thus identifying the best and most relevant targets.

In the case of rheumatoid arthritis, the disease state can be represented as outputs associated with, for example, enzyme activities, product formation dynamics, and cellular functions that can indicate one or more biological processes that cause, affect, or are modified by the disease state. Typically, the outputs of the computer model include a set of values that represent levels or activities of biological constituents or any other behavior of the disease state. Based on these outputs, one or more biological processes can be designated as critical biological processes.

The computer model can be executed to represent a modification to one or more biological processes, as described in greater detail in co-pending application, U.S. Ser. No. 10/938,072 filed Sep. 10, 2004. In particular, a modification to a biological process can be represented in the computer model to identify the degree of connection (e.g., the degree of correlation) between the biological process and rheumatoid arthritis. For example, a modification to a biological process can be represented in the computer model to identify the degree to which the biological process causes, affects, or is modified by rheumatoid arthritis. A biological process can be identified as causing rheumatoid arthritis if a modification to this biological process is observed to produce symptoms associated with rheumatoid arthritis, i.e., increased synovial cell density, cartilage degradation, bone erosion and IL-6 levels in the synovial tissue. In some instances, a modification to a biological process can be represented in the computer model to identify the degree of connection between other biological processes and rheumatoid arthritis.

In some instances, identifying the set of biological processes can include sensitivity analysis. Sensitivity analysis can involve prioritization of biological processes that are associated with the disease state and can be performed with different configurations of the computer model to determine the robustness of the prioritization. In some instances, sensitivity analysis can involve a rank ordering of biological processes based on their degree of connection to the disease state. Sensitivity analysis allows a user to determine the importance of a biological process in the context of the disease state. An example of a biological process of greater importance is a biological process that increases the severity of the disease state. Thus, inhibiting this biological process can decrease the severity of the disease state. The importance of a biological process can depend not only on the existence of a connection between that biological process and the disease state but also on the extent to which that biological process has to be modified to achieve a change in the severity of the disease state. In a rank ordering, a biological process that plays a more important role in the disease state typically gets a higher rank. The rank ordering can also be done in a reverse manner, such that a biological process that plays a more important role in the disease state gets a lower rank. Typically, the set of biological processes include biological processes that are identified as playing a more important role in the disease state.

During the process of sensitivity analysis of rheumatoid arthritis the activity of biological processes such as but not limited to monocytes recruitment, T-cell recruitment, cell apoptosis, and cytokine production are modulated (increased and decreased) in a computer model one a time. Biosimulation is then conducted and the consequence of the modulation of a single biological process at different level of stimulation or inhibition is assessed by measuring clinical outcomes such as, but not restricted to, cartilage degradation, synovial cell density, bone erosion and IL-6 levels. The outcome of this analysis identified the biological processes that have significant impact on the clinical outcomes.

In the present invention, sensitivity analysis identified three areas of the biology of rheumatoid arthritis having a significant impact on the disease pathophysiology: (1) monocyte/macrophage and T-cell recruitment, (2) monocyte/macrophage and T-cell apoptosis, and (3) macrophage cytokine (especially, TNF-α, IL-1 and IL-10) production.

1. Target Identification

We have discovered, based on the effects of HIF-1α activity inhibition by the model described above, blockade of HIF-1α activity is predicted to be an effective therapy for rheumatoid arthritis.

The effects of HIF-1α activity on monocyte/macrophage and T-cell recruitment, macrophage and T-cell apoptosis, and macrophage cytokine (particularly, TNFα, IL-1 and IL-10) production were quantified and explicitly represented in a computer model of rheumatoid arthritis. As the contribution of HIF-1α activity on each of these biological processes is not precisely quantified, a range of effects was defined in order to characterize the contribution of HIF-1α activity (Table 1). The “lower max effect” value represents the lowest documented effect taking in consideration possible redundancies with other proteins, the “upper max effect” is the maximal effect of HIF-1α activity on each biological process and the “most likely max effect” is the estimation of the realistic contribution of HIF-1α activity in each biological process, taking in consideration the in vivo environment and probable redundancies with other proteins.

TABLE 1
Effect of HIF-1α Activity on Joint Model

| | Lower | Most likely | Upper |
|---|---|---|---|
| Hypothesis | max effect | max effect | max effect |
| monocyte recruitment | 0.8x | 0.5x | 0.4x |
| T-cell recruitment | 0.8x | 0.8x | 0.4x |
| monocyte/macrophage | 1x | 1.5x | 3x |
| and T-cell apoptosis | | | |
| cytokine production | 0.7x | 0.6x | 0.3x |

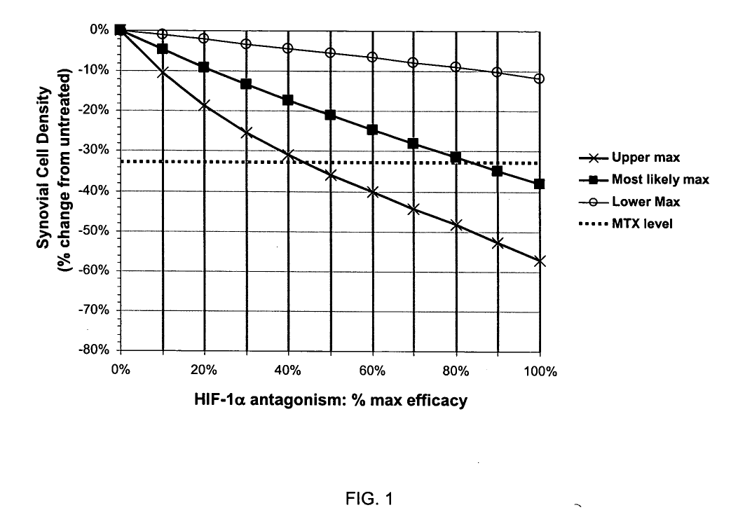

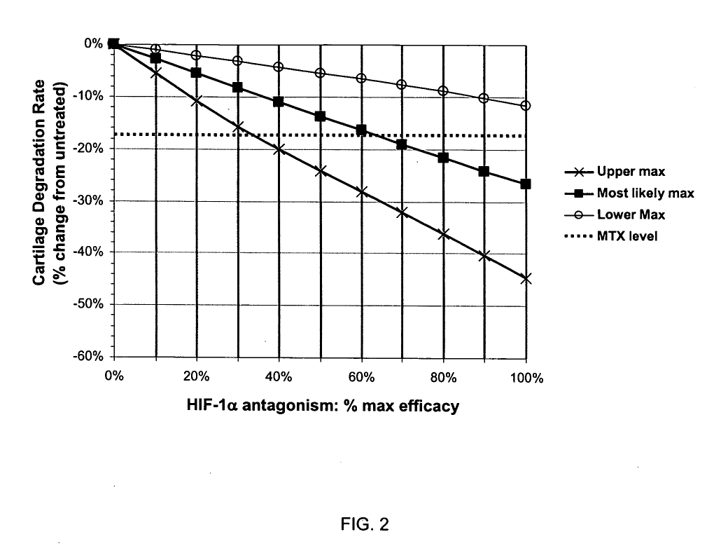

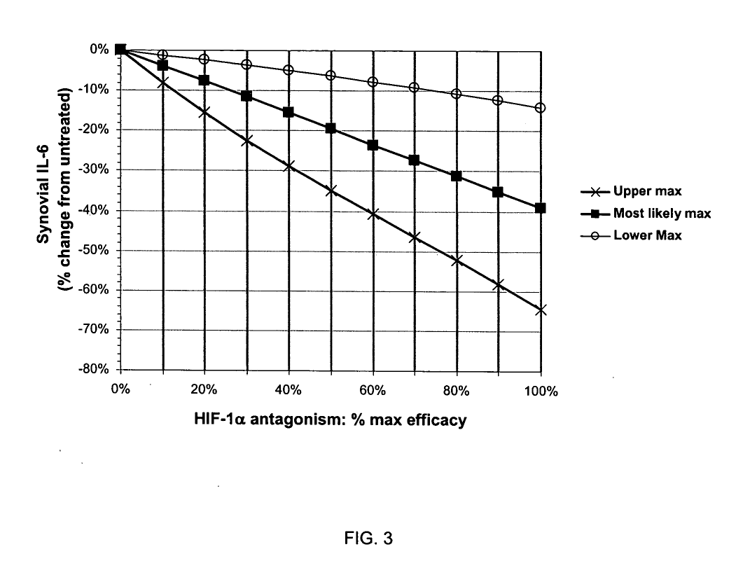

Simulation of the effect of HIF-1α activity on rheumatoid arthritis was then conducted by blocking HIF-1α in all relevant biological processes at once or in one biological process at time or in several biological processes in combination. The results of the simulation showed that blocking HIF-1α activity for 6 months could improve the rheumatoid arthritis clinical outcome by reducing cartilage degradation by 12 to 45%, synovial cell hyperplasia by 12 to 57% and IL-6 levels in synovial tissue by 14 to 65%. FIG. 1 demonstrates the effect of HIF-1α blockade on synovial cell density. FIG. 2 demonstrates the effect of HIF-1α blockade on cartilage degradation. FIG. 3 demonstrates the effect of HIF-1α blockade on IL-6 levels in synovial tissue.

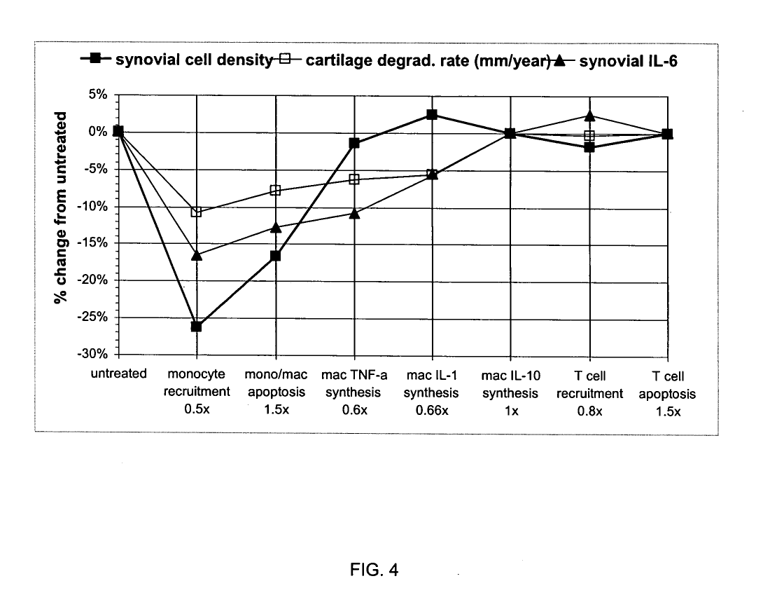

The simulation of HIF-1α blockade in one biological process at a time demonstrated that the main biological process driving the impact of HIF-1α blockade on the clinical outcome is the effect on monocyte recruitment. The impact of HIF-1α blockade on macrophage apoptosis also plays a significant role in improvements in the clinical markers of rheumatoid arthritis. FIG. 4 provides the response of three key therapeutic indices in a typical rheumatoid arthritis patient upon simulation of the effect of HIF-1α blockade on monocyte and T-cell recruitment, monocyte/macrophage and T-cell apoptosis, and macrophage cytokine production, independently.

Methotrexate is a common treatment for rheumatoid arthritis. Methotrexate treatment is known to decrease synovial cell density by approximately 30%, decrease the rate of cartilage degradation by approximately 15% and decrease the concentration of IL-6 in synovial tissue by 93%. At 100% efficacy, the computer model predicts HIF-1α antagonism is most likely to induce a greater improvement than methotrexate in these three therapeutic indices. The model predicts that compounds causing only 80% inhibition of HIF-1α activity would be superior to methotrexate in decreasing synovial cell density and the rate of cartilage degradation.

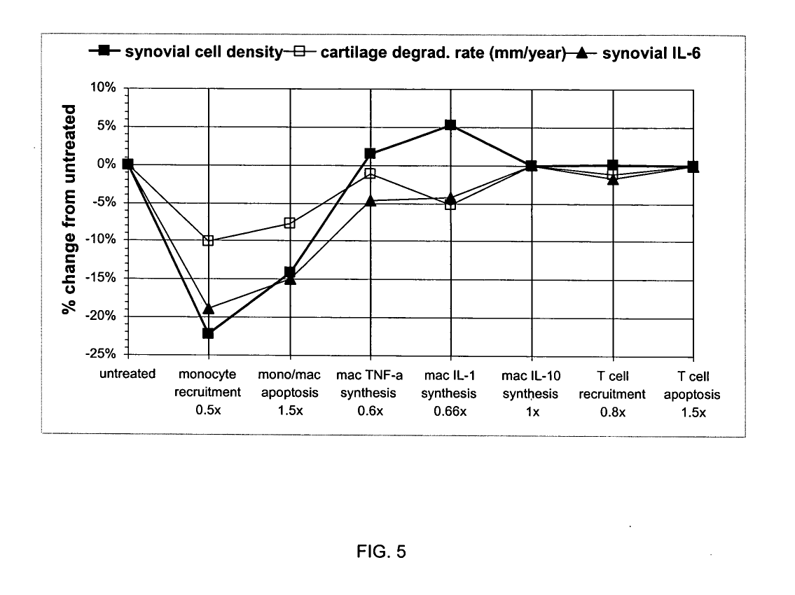

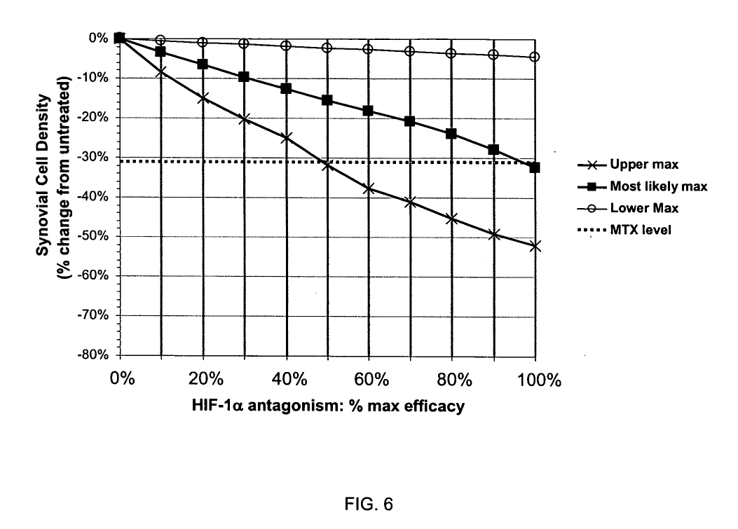

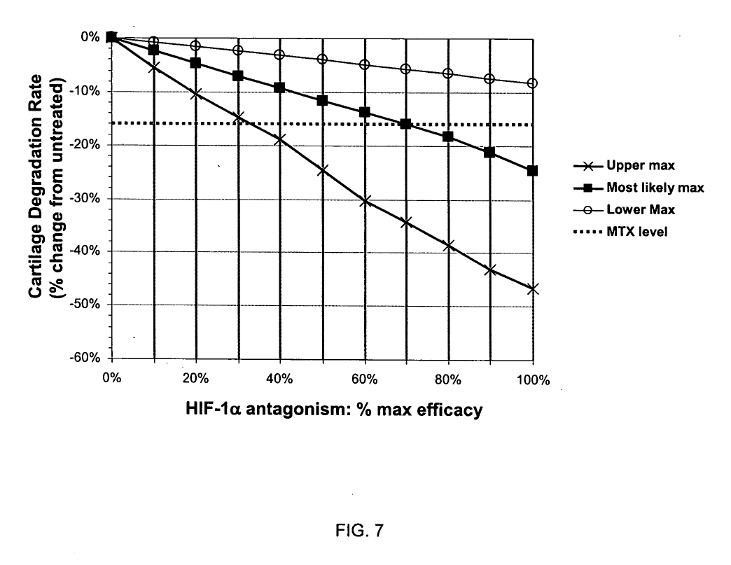

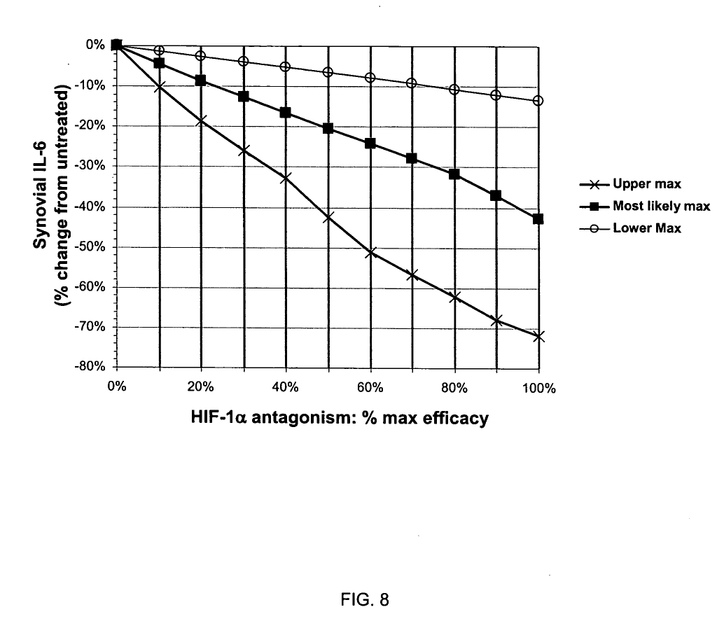

Some rheumatoid arthritis patients do not effectively respond to methotrexate treatment (initial non-responders), while other patients who initially responded to methotrexate become refractory over time (gradual non-responders). Both types of patients are referred to as methotrexate resistant patients. Simulation of blockading HIF-1α activity in a methotrexate resistant patient reveals a similar pattern of response than in a non-resistant patient. FIG. 5 provides the response of three key therapeutic indices in a methotrexate resistant patient upon simulation of the effect of HIF-1α blockade on monocyte and T-cell recruitment, monocyte/macrophage and T-cell apoptosis, and macrophage cytokine production, independently. Blocking HIF-1α activity for 6 months in a methotrexate resistant patient could improve the rheumatoid arthritis clinical outcome by reducing cartilage degradation by 8 to 47%, synovial cell hyperplasia by 5 to 52%, and IL-6 concentration by 13 to 73%. FIG. 6 demonstrates the effect of HIF-1α blockade on synovial cell density in a methotrexate resistant patient. FIG. 7 demonstrates the effect of HIF-1α blockade on cartilage degradation in a methotrexate resistant patient. FIG. 8 demonstrates the effect of HIF-1α blockade on IL-6 concentration in a methotrexate resistant patient.

Application of the in silico model of rheumatoid arthritis provided the surprising result that antagonism of HIF-1α activity is a promising therapeutic strategy for patients suffering from rheumatoid arthritis.

2. Thresholds

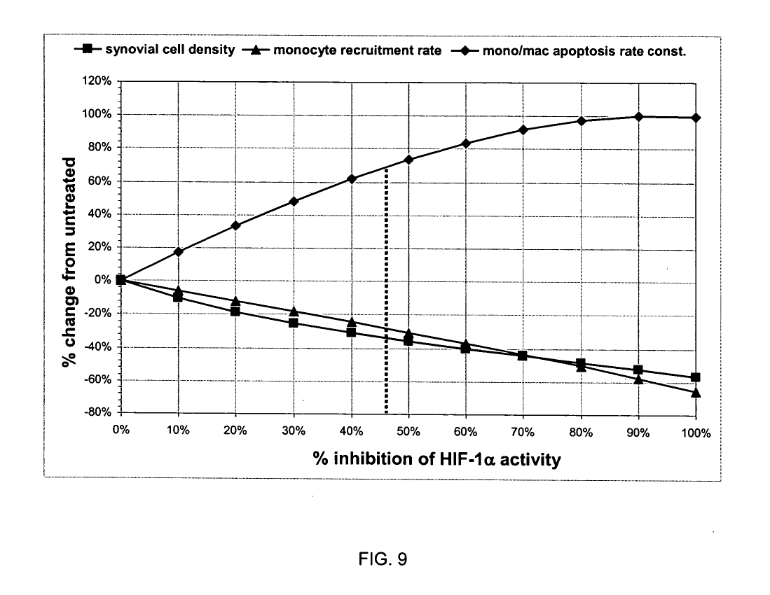

Although the amount of HIF-1α inhibition is correlated to decreased monocyte/macrophage recruitment and increased macrophage apoptosis, the alterations in recruitment rate and apoptosis rate are not linearly related to HIF-1α inhibition. FIG. 9 provides a comparison of HIF-1α inhibition with expected increase of macrophage apoptosis and decreased monocyte recruitment rates at the ‘upper maximum effect’ of HIF-1α antagonism. Each of these rates is compared to the therapeutic index of synovial cell density. The model found that to achieve a significant improvement in rheumatoid arthritis symptoms (i.e., at least 30% decrease in synovial cell density) in the reference patient, macrophage apoptosis must increase by at least approximately 1.7-fold and the rate of monocyte recruitment must decrease by at least approximately 30% after 24 hours of HIF-1α blockade. Such a level of HIF-1α blockade should result in approximately a 25% reduction in angiogenesis.

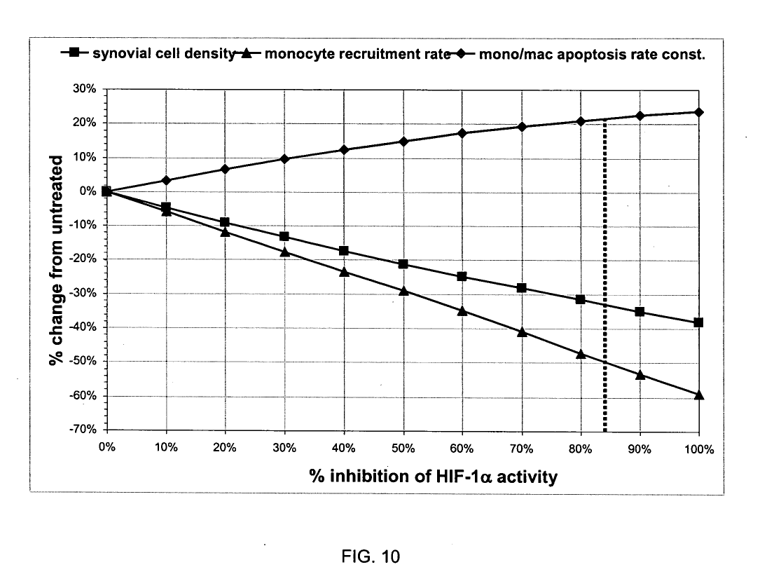

FIG. 10 provides a comparison of HIF-1α inhibition with expected increase of macrophage apoptosis and decreased monocyte recruitment rates at the ‘most likely maximum effect’ of HIF-1α antagonism. Each of these rates is compared to the therapeutic index of synovial cell density. The model found that to achieve a significant improvement in rheumatoid arthritis symptoms (i.e., at least 30% decrease in synovial cell density) in the reference patient, macrophage apoptosis must increase by at least approximately 1.25-fold and the rate of monocyte recruitment must decrease by at least approximately 50% after 24 hours of HIF-1α blockade. In view of both hypotheses, the global threshold for therapeutic antagonism of HIF-1α activity would result in at least a 1.5-fold increase in macrophage apoptosis and in at least a 40% decrease in monocyte recruitment to the synovium. Such a level of HIF-1α blockade should result in approximately a 50% reduction in angiogenesis.

D. HIF-1α

HIF-1α is a transcription factor that plays a central role in the control of cellular adaptation to hypoxic conditions, such as occur in the rheumatic joint. The synovium is physiologically a hypoxic environment. Oxygen levels become even lower in the joint during rheumatoid arthritis inflammation characterized by high levels of lactate, and somewhat surprisingly, of VLDL, LDL, and HDL. Treuhaft and McCarty, Arthritis Rheum. 14: 475-84 (1971); and Naughton, et al., FEBS Lett 332: 221-225 (1993). Cellular adaptation to this environment is thought to promote persistent inflammation.

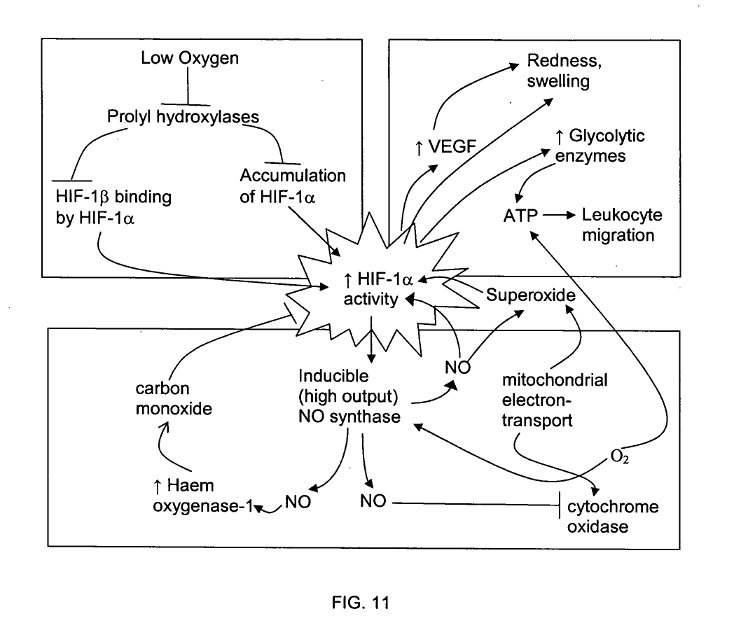

Hypoxia-inducible factor 1 (HIF-1) is a heterodimeric protein that consists of two subunits—HIF-1α and HIF-1β. HIF-1α is a member of the basic helix-loop-helix-PAS transcriptional factor family. HIF-1α has a molecular weight of 6959da and is composed of 826 amino-acid residues and four functional domains. HIF-1 activates the transcription of many genes coding for proteins involved in angiogenesis, glucose metabolism, cell proliferation/survival and invasion/metastasis. Hypoxia regulates the level of HIF-1α protein by inhibiting its ubiquitin-mediated degradation (FIG. 11). See, Semenza, “Targeting HIF-1 for Cancer Therapy,” Nat Rev Cancer 3: 721-732 (October 2003).

Originally described twenty years ago as regulator of hypoxia-induced expression of erythropoietin, the transcription factor HIF-1 has been shown to regulate an increasing number of genes (Table 2) in response to changes in tissue oxygenation and also in response to growth factor stimulation. Semenza (2003).

TABLE 2
Genes Transcriptionally Activated by HIF-1

| Function | Genes |
|---|---|
| Cell proliferation | Cyclin G2, Insulin growth factor (IGF)-2, |
| | IGF-binding proteins 1/2/3, WAF1, TGF-α, |
| | TGF-β3 |
| Cell survival, | Adrenomedullin, erythropoietin, IGF-2, |
| | IGF-binding proteins 1/2/3, Nitric oxide |
| | synthetase-2, TGF-α, Vascular endothelial |
| | growth factor (VEGF) |
| Apoptosis | NIP3, NIX, RTP801 |
| Motility | Autocrine motility factor/GPI, c-MET, LDL |
| | receptor-related protein 1, TGF-α |
| Cytoskeletal structure | Keratin 14/18/19, Vimentin |
| Cell adhesion | MIC2/CD99 |
| Erythropoiesis | Erythropoietin |
| Angiogenesis, | Endocrine gland derived VEGF, Endoglin, |
| | Leptin, LDL receptor-related protein 1, |
| | TGF-β3, VEGF |
| Vascular tone, | α_{1B}-adrenergic receptor, Adrenomedullin, |
| | Endothelin-1, Haem oxygenase-1, Nitric oxide |
| | synthetase-2 |
| Transcriptional | DEC1, DEC2, ETS-1, NUR77 |
| regulation | |
| pH regulation | Carbonic anhydrase 9 |
| Epithelial homeostasis | Intestinal trefoil factor |
| Drug resistance | Multidrug resistance 1 |
| Nucleotide metabolism | Adenylate kinase 3, Ecto-5′-nucleotidase, |
| Iron metabolism | Ceruloplasmin, Transferrin, Transferrin |
| | receptor |
| Glucose metabolism | Hexokinase 1/2, Autocrine motility factor /GPI, |
| | Enolase 1, Glucose transporter 1, GAPDH, |
| | Lactate dehydrogenase, 6-phosphofructo-2- |
| | kinase/fructose-2,6-biphosphatase-3, |
| | Phosphofructokinase L, Phosphoglycerate |
| | kinase1, Pyruvate kinase M, Triosephosphate |
| | isomerase |
| Extracellular-matrix | Cathepsin D, Collagen type V (α1) , |
| metabolism | Fibronectin 1, Matrix metalloproteinase 2, |
| | Plasminogen-activator inhibitor 1, |
| | Prolyl-4-hydroxylase α (I), Urokinase |
| | plasminogen activator receptor |
| Energy metabolism | Leptin |
| Amino-acid metabolism | Transglutaminase 2 |

HIF-1α protein synthesis is regulated by activation of the phosphatidylinositol 3-kinase (PI3K) and ERK mitogen-activated protein kinase (MAPK) pathways. These pathways can be activated by signaling via receptor tyrosine kinases, non-receptor tyrosine kinases or G-protein-coupled receptors.

Hypoxia regulates HIF-1α at the level of protein stability by inhibiting its ubiquitin-mediated degradation. HIF-1α protein degradation is regulated by O₂-dependent prolyl hydroxylation, which targets the protein for ubiquitylation by E3 ubiquitin-protein ligases. Prolyl hydroxylase uses molecular oxygen as a substrate and thus provides a direct measure of the availability of oxygen. The E3 ubiquitin-protein ligases contain the von Hippel-Lindau tumor-suppressor protein (VHL), which binds specifically to hydroxylated HIF-1α. Ubiquitylated HIF-1α is rapidly degraded by the proteasome.

HIF-1α is known to be overexpressed in many human cancers as a result of intratumoral hypoxia as well as genetic alterations, such as gain-of-function mutations in oncogenes (i.e., ERBB2) and loss-of-function mutations in tumor-suppressor genes (i.e., VHL and PTEN). While the activity of HIF-1α has been correlated with tumor survival, antagonism of HIF-1α activity, particularly at the thresholds defined by the present invention, has not been linked with the pathology of rheumatoid arthritis.

E. Methods of Identifying HIF-1α Antagonists and Anti-Rheumatic Drugs

1. Monocyte Recruitment

As described above, inhibiting monocyte/macrophage recruitment is a major contributor to the benefits of HIF-1α blockade. One preferred assay for identifying antagonists of HIF-1α activity is a modification of a typical transmigration assay. Monocytes are in suspension above an endothelial layer growing on a porous support above a lower well of endogenous (made by the endothelium) or exogenous chemoattractant. The monocytes that end up in the lower chamber at the end of the assay are counted as transmigrated. Compounds that inhibit the activity of HIF-1α will decrease the number of cells that migrate across the endothelial layer.

In one preferred assay, endothelial cells are cultured on hydrated Type I collagen gels overlaid with fibronectin. Components of the culture medium penetrate into the porous gel. Alternatively, the endothelial cells may be grown on the upper surface of a porous filter suspended above a lower chamber. Culture medium is placed in the upper and lower chambers to reach the apical and basal surfaces of the monolayer. Monocytes are added to the upper chamber. In order to be counted as “migrated”, a monocyte must (1) attach to the apical surface of the endothelial cells, (2) migrate to the intercellular junction, (3) diapedese between the endothelial cells, (4) detach from the endothelial cells and penetrate the basal lamina, (5) cross the filter or gel and (6) detach from the filter or gel and enter the lower chamber.

Monocytes or neutrophils, freshly isolated from peripheral blood of healthy or rheumatic donor are allowed to settle on confluent endothelial monolayers at 37° C. in the presence or absence of test compounds. The assays may be run in a variety of media including, but not limited to complete medium, Medium 199, or RPMI1640, optionally supplemented with human serum albumin. After sufficient time for transendothelial migration, generally one hour, the monolayers are washed with a chelator, such as EGTA, to remove any monocytes or neutrophils still attached to the apical surface. If a collagen gel is used as a substrate, the monolayer is then rinsed with phosphate buffered saline with divalent cations and fixed in glutaraldehyde overnight. Fixing strengthens the collagen gel so that it is easier to manipulate. The monolayers are stained, preferably with Wright-Giemsa, and mounted on slides for direct observation, preferably under Nomarski optics. Using Nomarski optics, one can distinguish by the plane of focus, monocytes or neutrophils that are attached to the apical surface of the monolayer from those that have transmigrated. A quantifiable measure of transmigration is the percentage of those monocytes or neutrophils associated with the monolayer that have migrated beneath the monolayer. Therefore, the measurement of transmigration is independent of the degree of adhesion to the monolayer.

HIF-1α has been demonstrated to regulate the level of expression of hypoxia-responsive genes in macrophages under both hypoxic and normoxic conditions. Cramer, et al., Cell 112: 645-657 (2003). Therefore, the rate of monocyte migration can be evaluated under either hypoxic or normoxic conditions. Preferably, the assays will be performed under hypoxic conditions.

Migration of monocytes or neutrophils can be determined in the presence or absence of cytokine stimulation of the endothelium. Activation of endothelial cells can result from contact with stimulatory mediators and typically will enhance migration of monocytes or neutrophils across the endothelium. For the purpose of the present invention, activation of endothelial cells preferably results from contact with cytokines such as tumor necrosis factor (TNF) and interleukin-1 (IL-1).

The term “endothelial cell” has ordinary meaning in the art. Endothelial cells make up endothelium, which is found inter alia in the lumen of vascular tissue (veins, arteries, and capillaries) throughout the body. In arthritis, leukocytes migrate from the circulating blood to the arthritic joint where they participate in inflammation.

2. Monocyte/Macrophage and T-cell Apoptosis

As described above, inhibiting monocyte/macrophage apoptosis is the second major contributor to the expected benefits of HIF-1α blockade. Apoptosis measurement can vary depending on the cell type and the assay used. It may be advantageous to use a combination of standard apoptotic assays (e.g., Annexin V or TUNEL assays) to measure the percentage of apoptotic monocytes/macrophages and a quantitative anti-histone ELISA to measure the global effect of HIF-1α blockade on apoptosis. The amount of macrophage apoptosis can be evaluated under either hypoxic or normoxic conditions. Preferably, the assays will be performed under hypoxic conditions.

a. DNA Fragmentation Assays

Loss of DNA integrity is a characteristic of apoptosis. When DNA extracted from apoptotic cells is analyzed using gel electrophoresis, a characteristic “ladder” of DNA fragments is seen. However, extraction of DNA from cells is a time consuming process and alternative methods are equally suitable for detecting the characteristic fragmentation of DNA in apoptotic cells. DNA fragmentation can be detected by a variety of assay including propidium iodide assays, acridine orange/ethidium bromide double staining, TUNEL and ISNT techniques, and the assays of DNA sensitivity to denaturation.

b. Annexin V Assays

Externalization of phosphatidylserine (PS) and phosphatidylethanolamine is a hallmark of the changes in the cell surface during apoptosis. Annexin V is a 35-36 kDa Ca²⁺-dependent, phospholipid binding protein that has a high affinity for PS and binds to cells with exposed PS. Annexin V may be conjugated to any of a variety of markers to permit it to be detected by microscopy or flow cytometry. For use in methods of identifying compounds the inhibit HIF-1α activity or methods of screening for compounds that inhibit HIF-1α activity, it is preferable to use fluorescently labeled annexin V detected by flow cytometry.

Macrophages are obtained as discussed above from either rheumatoid or healthy subjects. Cells are incubated with the test compound for one to 24 hours, optionally in the presence of a death receptor-dependent inducer of apoptosis. The number of cells committed to apoptosis is determined by staining with labeled annexin V and a vital dye, such as propidium iodide (PI) or 7-amino-actinomycin D (7-AAD). Because externalization of PS occurs in the earlier stages of apoptosis, annexin V staining precedes the loss of membrane integrity which accompanies the latest stages of cell death resulting from either apoptotic or necrotic processes. Therefore, staining with annexin V in conjunction with vital dyes such as propidium iodide (PI) or 7-amino-actinomycin D (7-AAD) permits identification of early apoptotic cells (annexin V-positive and vital dye-negative).

3. HIF-1α Expression

The activity of HIF-1α can be antagonized by decreasing the expression of HIF-1α or by increasing the proteolytic degradation of expressed HIF-1α. Methods of determining expression levels of proteins are well known in the art. Any measurement technique, now known or discovered in the future, may be used to determine the amount of HIF-1α protein that is expressed or present in a cell. The method exemplified herein it just one of the many acceptable methods for determining HIF-1α expression levels.

Monocytes or macrophages can be isolated from synovial fluid or peripheral blood mononuclear cells from rheumatoid arthritis patients or healthy donors by either Percoll or Histopaque (Sigma Chemical Co.) gradient centrifugation or countercurrent centrifugal elutriation (Beckman-Coulter). Monocytes can be differentiated into macrophages with RPMI containing 20% heat-inactivated fetal bovine serum (FBS) plus 1 μg/ml polymyxin B sulfate (Sigma Chemical Co.) in 24-well plates (Costar). The macrophages are incubated with a compound of the invention for periods of time ranging from one hour to several days. After incubation, the cells are lysed by any suitable method to produce a cell lysate. The amount HIF-1α expression can be determined via Western Blot, immunoprecipitation or any other quantitative procedure utilizing anti-HIF-1α antibodies. Suitable anti-HIF-1α antibodies include polyclonal and monoclonal antibodies (clone OZ12, OZ15, Hla67, ESEE122). Any antibody or antibody fragment, polyclonal or monoclonal antibody specific for HIF-1α may be used to quantify HIF-1α expression. Appropriate negative controls, including cells treated identically to the test cells with the exception of exposure to the test compound should be performed in order to identify alterations in HIF-1α expression due to exposure to the compound rather than manipulations of the cells during experimentation.

Various procedures, well known in the art, may be used for the production of polyclonal antibodies to HIF-1α. For example, for the production of polyclonal antibodies, various host animals, including but not limited to rabbits, mice, rats, etc., can be immunized by injection with HIF-1α or a derivative thereof. Various adjuvants may be used to increase the immunological response, depending on the host species, and including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

A monoclonal antibody (mAb) to HIF-1α can be prepared by using any technique known in the art which provides for the production of antibody molecules by continuous cell lines in culture. These include but are not limited to, the hybridoma technique originally described by Kohler and Milstein (Nature 256: 495-497 (1979)), the more recent human B cell hybridoma technique (Kozbor et al., Immunology Today 4: 72 (1983)), and the EBV-hybridoma technique (Cole et al., 1985, Monoclonal Antibodies and Cancer Therapy, Alan R. Liss, Inc., pp. 77-96). Such antibodies may be of any immunoglobulin class including IgG, IgM, IgE, IgA and, IgD and any subclass thereof. The hybridoma producing the mAbs of use in this invention may be cultivated in vitro or in vivo.

4. HIF-1α Transcriptional Activation

The methods of the invention also contemplate directly inhibiting the transcriptional activation activity of HIF-1α. The transcriptional activation activity of HIF-1α can be determined either by examining the amount of HIF-1α protein bound to its target hypoxia-responsive element (HRE) or by examining the amount of HIF-1α-inducible expression of a hypoxia-responsive gene, such as VEGF. The interaction between HIF-1α and an HRE can be determined by any standard protein-DNA interaction assay, known at present or discovered in the future. The preferred sequence of the HRE is sequence 5′-TACGTGCT-3′ (SEQ ID NO: 1). In a preferred method, the amount of transcription from an HIF-1α-responsive gene can be measured directly by determining the amount of an HIF-1α-responsive gene product, e.g., VEGF or erythropoietin. Alternatively, the promoter of an HIF-1α-responsive gene may be placed so as to control the expression of an easily detectable protein, such as luciferase or green fluorescent protein.

5. Angiogenesis

The activity of HIF-1α plays an important role in angiogenesis. Therefore, inhibition of HIF-1α activity can be demonstrated by inhibition of angiogenesis. Several methods of determining angiogenic activity are known in the art. Any measurement technique, now known or discovered in the future, may be used to measure the effect of an antagonist of HIF-1α activity on angiogenesis. In an exemplary method, endothelial cells are incubated on a solid gel of basement proteins (available as ECMatrix™ from Chemicon, Intl., Temecula, Calif.). The basement membranes can include a large number of relevant proteins such as laminin, collagen type IV, heparan sulfate proteoglycans, entactin, nidogen, growth factors, (such as, TGFβ and FGF), and proteolytic enzymes (such as, plasminogen, tPA, and MMPs). Any source of endothelial cells may be used. Human umbilical vein endothelial cells (HUVEC) are a preferred source of endothelial cells for the assays of the invention. The endothelial cells are incubated with the basement proteins, in the presence or absence of the test compound for 4-18 hours. The formation of cellular networks, an indication of capillary formation can be identified by a variety of methods including visualizing cell tubes, counting branch points or calculating the total capillary length in a defined view-field area. Staining of the cells with agents such as crystal violet can ease identification of capillary formation.

F. Methods of Treatment

In one aspect, the invention provides methods of alleviating at least one symptom of an inflammatory disease, such as rheumatoid arthritis, comprising administering a therapeutically effective amount of an antagonist of HIF-1α activity to a patient having an inflammatory disease. The invention also provides methods for alleviating at least one symptom of rheumatoid arthritis comprising administering a therapeutically effective amount of an antagonist of HIF-1α activity to a patient having rheumatoid arthritis. The antagonist of HIF-1α activity maybe a protein, nucleic acid or small molecule inhibitor. A preferred protein antagonist is an antibody, more preferably a monoclonal antibody. Preferred nucleic acid antagonists include antisense inhibitors of the gene encoding HIF-1α. The invention also encompasses methods of decreasing synovial cell density, methods of decreasing cartilage degradation and methods of decreasing IL-6 concentration in synovial tissue by administering a therapeutically effective amount of an antagonist of HIF-1α activity.

Antisense inhibitors have been shown to be capable of interfering with expression of target proteins. See Cohen, “Designing antisense oligonucleotides as pharmaceutical agents,” Trends Pharmacol Sci. 10: 435-7(1989) and Weintraub, “Antisense RNA and DNA,” Sci Am. 262: 40-6 (1990), both incorporated herein by reference. Antisense inhibitors of HIF-1α are described in detail in PCT publication WO 03/085110, incorporated by reference herein.

Small molecules have also been shown to be capable of inhibiting the activity of HIF-1α in the context of anti-cancer therapies. For example, small molecules can increase the activity of the prolyl hydroxylase enzymes which target HIF-1α for proteolytic degradation. Exemplary compounds are described in PCT publication WO 02/074981, incorporated herein by reference. Other compounds known to inhibit HIF-1α activity include YC-1 stimulator (5′-hydroxymethyl-2′-furyl)-1-benzylindazole), 2-methoxyestradiol, taxol, vincristine, 1-methylpropyl-2-imidazolyl disulphide, pleurotin, rapamycin/CC1779, LY294002, wortmannin, geldanamycin, camptothecin (and analogs, such as Topotecan), PD98059, and quinocarmycin (and analogs such as NCS-607097 (DX-52-1)). An exemplary protein inhibitor of HIF-1α activity is phosphate and tension homologue (PTEN).

A compound useful in this invention is administered to a patient in a therapeutically effective dose by a medically acceptable route of administration such as orally, parenterally (e.g., intramuscularly, intravenously, subcutaneously, intraperitoneally), transdermally, rectally, by inhalation and the like. The dosage range adopted will depend on the route of administration and on the age, weight and condition of the patient being treated.

Various delivery systems are known and can be used to administer a composition of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262: 4429-4432), construction of a nucleic acid as part of a retroviral or other vector, etc. Methods of introduction include, but are not limited to, intradermal, intramuscular, intraperitoneal, intravenous, subcutaneous, intranasal, epidural, and oral routes. The compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

G. Combination Therapies

In one aspect, the invention provides methods of alleviating at least one symptom of rheumatoid arthritis, comprising administering an antagonist of HIF-1α activity and an anti-inflammatory drug to a patient having rheumatoid arthritis. Preferably, the anti-inflammatory drug is selected from the group of methotrexate, a TNF-α antagonist, an interleukin-1 receptor antagonist and a steroid. More preferably, the anti-inflammatory drug is methotrexate, Anakinra or prednisone. In one embodiment of the invention, the patient is resistant to methotrexate or to TNF-α blockade.

Various treatment protocols were simulated alone, or in combination with antagonism of HIF-1α activity. The effects of several therapies are represented in the model. The model reproduces the impact of treatment with (1) non-steroidal anti-inflammatory drugs (NSAIDs; e.g., indomethacin), (2) Etanercept, a soluble type II TNF-α receptor, (3) methotrexate (MTX), (4) glucocorticoids (e.g., methylprednisolone), and (5) Anakinra, an IL-1 receptor antagonist (IL-1Ra).

Each therapy is implemented based on its mode of action, molecular activity, and pharmacokinetic properties as well as its recommended clinical dosing regimen. To determine the importance of time-dependent variation in drug exposure associated with the clinically recommended periodic drug administration, we compared simulation results based on the clinical schedule with results for a constant-concentration continuous dose with an equivalent serum area-under-the-curve (AUC) net drug exposure. Simulation results for the two different administration schedules differed only minimally. In order to simplify presentation of results by eliminating transient effects due to periodic administration, results discussed herein are based on continuous dose therapy simulations.

The impact of the simulated treatments results from the implemented molecular activity. For example, Etanercept is modeled as binding and neutralizing TNF-α; any subsequent changes in hyperplasia, cartilage degradation, or other measurements are a secondary consequence of this reduction in free, active TNF-α, rather than a direct or specified effect of Etanercept. The effects directly implemented for each therapy are as follows:

The primary, common mode of action of NSAIDs is the inhibition of the cyclo-oxygenase (COX) pathways and synthesis of their downstream products, especially prostaglandin-E2. The model implementation of NSAIDs is based on in vitro data on the dose-dependent inhibition by NSAIDs of PGE2 synthesis in macrophages, FLS, and chondrocytes. Simulation results presented are for a constant continuous dose with serum AUC drug exposure equivalent to that achieved with a dosing schedule of 50 mg indomethacin, administered orally 3 times a day.

Etanercept and Infliximab (exogenous sTNF-RII and anti-TNF-α antibody respectively) are modeled as binding and neutralizing soluble TNF-α. The binding of these agents to TNF-α is modeled using appropriate values for binding rate parameters of each molecule. The net binding rate of soluble receptors (or anti-TNF-α) to TNF-α is calculated as the difference between the binding and dissociation rates as follows:

$$\frac{ⅆ}{ⅆ t} [ TNF α \text{:} sTNFR ] = {k}_{on} [ TNF α ] [ sTNFR ] - {k}_{off} [ TNF α \text{:} sTNFR ] \\ where \\ {k}_{on} = constant of association between sTNF \text{-} R and TNF \text{-} α \\ {k}_{off} = constant of dissociation between sTNF \text{-} R and TNF \text{-} α \\ [ TNF α ] = concentration of free TNF \text{-} α \\ [ sTNFR ] = concentration of free soluble TNF \text{-} R \\ [ TNF α \text{:} sTNFR ] = concentration of bound complexes \qquad ( eq . 1 )$$

Simulation results presented are for a constant continuous dose of Etanercept with serum AUC drug exposure equivalent to that achieved with a dosing schedule of 25 mg, administered subcutaneously twice a week.

Methotrexate therapy is implemented based on in vitro data that quantify its direct effects on particular cellular functions, including dose-dependent inhibition of T cell and FLS proliferation, mediator synthesis, and apoptosis. In addition, to account for the inhibitory effect of methotrexate on vascular proliferation and vascularization, a reduction in total endothelial adhesion molecules expression is also implemented. Simulation results presented are for a constant continuous dose with serum AUC drug exposure equivalent to that of a dosing schedule of 12.5 mg/week, administered orally.

Methylprednisolone is represented by the dose-dependent modulation of various cellular mediator synthesis rates according to in vitro data. Effects on other cell functions are not directly modeled but may arise from altered mediator-dependent regulation. Simulation results presented are for a constant continuous dose with serum AUC drug exposure equivalent to that of a dosing schedule of 5 mg methylprednisolone, administered orally once a day.

Anakinra, like endogenous IL-1Ra, is modeled as reducing the impact of IL-1β on all cellular functions. This is implemented by calculating an “effective” IL-1 β concentration that has been adjusted to account for the impact of reduced receptor binding in the presence of the instantaneous concentration of receptor antagonist. Simulation results presented are for a constant continuous dose with serum AUC drug exposure equivalent to that of a dosing schedule of 100 mg Anakinra, administered subcutaneously once a day.

Simulation of the effect of treatment on the progression of rheumatoid disease in a virtual patient was conducted by simulating rheumatoid arthritis in the virtual patient for one year without treatment to establish a baseline in the model. Then either no treatment, a current treatment protocol or a current protocol in combination with HIF-1α antagonism was modeled. HIF-1α antagonism was modeled assuming 100% inhibition of HIF-1α activity having (i) the “upper max effect,” which represent maximal expected effect of HIF-1α activity on each biological process (ii) the “most likely max effect,” which is the estimation of the realistic contribution of HIF-1α activity, taking into consideration the in vivo environment and redundancies; and (iii) the “lower max effect,” which represents the lowest documented effect taking in consideration possible redundancies with other proteins. The effects of the simulated treatment (or lack of treatment) in a typical patient for six months on synovial cell density and cartilage degradation rate are shown in TABLE 3.

TABLE 3
Effects of HIF-1α Antagonism in Combination with Other Therapies

| | | | | MTX | | TNF | |
|---|---|---|---|---|---|---|---|
| | | Reference | | resistant | | non- | |
| | | patient | | patient | | responder | |
| First agent | Second agent | s.c.d. | c.d.r. | s.c.d. | c.d.r. | s.c.d. | c.d.r. |
| None | None | 100 | 100 | 100 | 100 | 100 | 100 |
| | anti-HIF-1α (lower maximum effect) | 88 | 88 | 96 | 92 | 89 | 88 |
| | anti-HIF-1α (most likely maximum effect) | 59 | 73 | 64 | 72 | 60 | 72 |
| | anti-HIF-1α (upper maximum effect) | 35 | 48 | 46 | 48 | 36 | 47 |
| NSAID | None | 103 | 105 | 105 | 106 | 104 | 106 |
| | anti-HIF-1α (lower maximum effect) | 91 | 92 | 98 | 95 | 92 | 92 |
| | anti-HIF-1α (most likely maximum effect) | 61 | 74 | 73 | 78 | 62 | 73 |
| | anti-HIF-1α (upper maximum effect) | 36 | 48 | 49 | 50 | 39 | 49 |
| Methotrexate | None | 67 | 82 | 81 | 87 | 97 | 100 |
| | anti-HIF-1α (lower maximum effect) | 57 | 71 | 69 | 76 | 86 | 88 |
| | anti-HIF-1α (most likely maximum effect) | 43 | 58 | 51 | 57 | 59 | 71 |
| | anti-HIF-1α (upper maximum effect) | 28 | 37 | 40 | 35 | 36 | 47 |
| Etanercept | None | 51 | 67 | 71 | 81 | 88 | 76 |
| | anti-HIF-1α (lower maximum effect) | 47 | 62 | 66 | 74 | 59 | 64 |
| | anti-HIF-1α (most likely maximum effect) | 39 | 57 | 56 | 66 | 41 | 55 |
| | anti-HIF-1α (upper maximum effect) | 32 | 46 | 46 | 48 | 31 | 44 |
| Anakinra | None | 82 | 55 | 90 | 54 | 90 | 60 |
| | anti-HIF-1α(lower maximum effect) | 65 | 44 | 73 | 45 | 68 | 45 |
| | anti-HIF-1α (most likely maximum effect) | 45 | 34 | 51 | 36 | 47 | 35 |
| | anti-HIF-1α (upper maximum effect) | 29 | 24 | 43 | 29 | 29 | 24 |
| Steroid | None | 59 | 59 | 70 | 64 | 61 | 58 |
| | anti-HIF-1α (lower maximum effect) | 50 | 52 | 60 | 55 | 52 | 51 |
| | anti-HIF-1α (most likely maximum effect) | 38 | 43 | 50 | 47 | 41 | 44 |
| | anti-HIF-1α (upper maximum effect) | 27 | 27 | 41 | 34 | 27 | 26 |

s.c.d. = % of synovial cell density as compared to untreated patient
c.d.r. = % of cartilage degradation rate as compared to untreated patient

The results of the simulation in a typical rheumatoid arthritis patient showed that blocking HIF-1α activity in addition to administration of an interleukin-1 receptor antagonist, such as Anakinra, can improve the rheumatoid arthritis clinical outcome by reducing cartilage degradation by 56 to 76% and synovial cell hyperplasia by 35 to 71%. Similarly, treatment with HIF-1α inhibition in combination with methotrexate, Etanercept or a steroid, such as methylprednisolone, shows decreases in synovial cell hyperplasia and cartilage degradation that cannot be achieved with a monotherapy.

Simulation of HIF-1α antagonism in combination with standard anti-rheumatic treatments in a methotrexate resistant patient revealed a pattern of response similar to that in a normal methotrexate-responsive patient. The effects of the simulated treatment (or lack of treatment) in a methotrexate resistant patient for six months on synovial cell density is summarized in Table 3. The results of the simulation showed that blocking HIF-1α activity in addition to administration of an interleukin-1 receptor antagonist, such as Anakinra, can improve the rheumatoid arthritis clinical outcome by reducing cartilage degradation by 55 to 71% and synovial cell hyperplasia by 27 to 57%. Interestingly, a combination therapy comprising HIF-1α antagonism and administration of methotrexate to a methotrexate resistant patient can improve the rheumatoid arthritis clinical outcome by reducing cartilage degradation and synovial cell hyperplasia to a greater extent than achieved by HIF-1α antagonism or methotrexate treatment alone. As with a typical rheumatoid arthritis patient, treatment with HIF-1α antagonism in combination with Etanercept or a steroid, such as methylprednisolone, shows decreases in synovial cell hyperplasia and cartilage degradation that cannot be achieved with a monotherapy

TNF-α neutralizing therapies have become increasingly important in treating rheumatoid arthritis patients. However, roughly a third of all rheumatoid arthritis patients fail to achieve a clinically significant response to TNF-α neutralizing therapies. Three potential classes of TNF-α blockade resistant patients were defined in the model described above. Synovial hyperplasia and cartilage degradation are differentially affected when TNF-α varies within different ranges, leading to the identification of three nonresponder classes within the current model. Specifically, patients with low initial TNF-α activity show decreased synovial hyperplasia, but minimal reduction in cartilage degradation in response to TNF-α blockade (cartilage nonresponders, or CNRs), while patients with negligible initial TNF-α activity show poor response in both synovial hyperplasia and cartilage degradation (double nonresponders or DNRs). Alternatively, insufficient neutralization of TNF-α in patients with abnormally high or resistant levels of TNF-α activity yields improvement in cartilage degradation but poor response in hyperplasia (hyperplasia nonresponders or HNRs). Mechanistically, in patients with low levels of TNF-α, rheumatoid disease was perpetuated by increased activity of alternate macrophage activating pathways (e.g., CD40-ligation), reduced activity of anti-inflammatory cytokines (e.g., IL-10), and increased activity of degradation-promoting cytokines (e.g., IL-1β). Nonresponding patients also showed altered responses to other therapies such as IL-1Ra (data not shown).

Patients who fail to achieve a significant clinical response to TNF-α blockade represent a sizable subset of the rheumatoid arthritis population. Simulation of HIF-1α antagonism in combination with standard anti-rheumatic treatments in a TNF-α hyperplasia nonresponder revealed a slightly different pattern of response than in a normal methotrexate-responsive patient. The effects of the simulated treatment (or lack of treatment) in a TNF-α hyperplasia nonresponder for six months on synovial cell density and cartilage degradation is shown in Table 3. The results of the simulation showed that combination therapy comprising HIF-1α antagonism and administration of methotrexate to a TNF-α blockade resistant patient showed no improvement in clinical outcome as compared to HIF-1α antagonism alone. However, combination of HIF-1α antagonism with Etanercept, IL-1Ra or steroid treatment can result in less synovial cell hyperplasia and lower cartilage degradation rates as compared to the monotherapy or HIF-1α antagonism alone. Blocking HIF-1α activity in addition to administration of an interleukin-1 receptor antagonist, such as Anakinra, improves the rheumatoid arthritis clinical outcome by reducing cartilage degradation by 55 to 76% and synovial cell hyperplasia by 32 to 71%.

An antagonist of HIF-1α activity and another anti-rheumatoid drug are administered concurrently. “Concurrent administration” and “concurrently administering” as used herein includes administering an antagonist of HIF-1α activity and another disease modifying anti-rheumatoid drug in admixture, such as, for example, in a pharmaceutical composition or in solution, or as separate compounds, such as, for example, separate pharmaceutical compositions or solutions administered consecutively, simultaneously, or at different times but not so distant in time such that the antagonist of HIF-1α activity and other disease modifying anti-rheumatoid drug cannot interact.

Regardless of the route of administration selected, the antagonist of HIF-1α activity and other anti-rheumatoid drug are formulated into pharmaceutically acceptable unit dosage forms by conventional methods known to the pharmaceutical art. An effective but nontoxic quantity of the antagonist of HIF-1α activity and other anti-rheumatoid drug are employed in the treatment. The antagonist of HIF-1α activity and other anti-rheumatoid drug may be concurrently administered enterally and/or parenterally in admixture or separately. Parenteral administration includes subcutaneous, intramuscular, intradermal, intravenous, injection directly into the joint and other administrative methods known in the art. Enteral administration includes tablets, sustained release tablets, enteric coated tablets, capsules, sustained release capsules, enteric coated capsules, pills, powders, granules, solutions, and the like.

H. Pharmaceutical Compositions

An aspect of the invention provides methods of manufacturing a drug useful for treating rheumatoid arthritis in a warm-blooded animal. The drug is prepared in accordance with known formulation techniques to provide a composition suitable for oral, topical, transdermal, rectal, by inhalation, parenteral (intravenous, intramuscular, or intraperitoneal) administration, and the like. Detailed guidance for preparing compositions of the invention are found by reference to the 18^{th} or 19^{th} Edition of Remington's Pharmaceutical. Sciences, published by the Mack Publishing Co., Easton, Pa. 18040. The pertinent portions are incorporated herein by reference.

Unit doses or multiple dose forms are contemplated, each offering advantages in certain clinical settings. The unit dose would contain a predetermined quantity of an antagonist of HIF-1α activity calculated to produce the desired effect(s) in the setting of treating rheumatoid arthritis. The multiple dose form may be particularly useful when multiples of single doses, or fractional doses, are required to achieve the desired ends. Either of these dosing forms may have specifications that are dictated by or directly dependent upon the unique characteristic of the particular compound, the particular therapeutic effect to be achieved, and any limitations inherent in the art of preparing the particular compound for treatment of inflammatory disease.

A unit dose will contain a therapeutically effective amount sufficient to treat rheumatoid arthritis in a subject and may contain from about 1.0 to 1000 mg of compound, for example about 50 to 500 mg.

In a preferred embodiment, the drug of the invention is formulated in accordance with routine procedures as a pharmaceutical composition adapted for intravenous administration to human beings. Typically, pharmaceutical compositions for intravenous administration are solutions in sterile isotonic aqueous buffer. Where necessary, the pharmaceutical composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The drug of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The compound will preferably be administered orally in a suitable formulation as an ingestible tablet, a buccal tablet, capsule, caplet, elixir, suspension, syrup, trouche, wafer, lozenge, and the like. Generally, the most straightforward formulation is a tablet or capsule (individually or collectively designated as an “oral dosage unit”). Suitable formulations are prepared in accordance with a standard formulating techniques available that match the characteristics of the compound to the excipients available for formulating an appropriate composition.

The form may deliver a compound rapidly or may be a sustained-release preparation. The compound may be enclosed in a hard or soft capsule, may be compressed into tablets, or may be incorporated with beverages, food or otherwise into the diet. The percentage of the final composition and the preparations may, of course, be varied and may conveniently range between 1 and 90% of the weight of the final form, e.g., tablet. The amount in such therapeutically useful compositions is such that a suitable dosage will be obtained. Preferred compositions according to the current invention are prepared so that an oral dosage unit form contains between about 5.0 to about 50% by weight (% w) in dosage units weighing between 5 and 1000 mg.

The suitable formulation of an oral dosage unit may also contain: a binder, such as gum tragacanth, acacia, corn starch, gelatin; sweetening agents such as lactose or sucrose; disintegrating agents such as corn starch, alginic acid and the like; a lubricant such as magnesium stearate; or flavoring such a peppermint, oil of wintergreen or the like. Various other material may be present as coating or to otherwise modify the physical form of the oral dosage unit. The oral dosage unit may be coated with shellac, a sugar or both. Syrup or elixir may contain the compound, sucrose as a sweetening agent, methyl and propylparabens as a preservative, a dye and flavoring. Any material utilized should be pharmaceutically-acceptable and substantially non-toxic. Details of the types of excipients useful may be found in the nineteenth edition of “Remington: The Science and Practice of Pharmacy,” Mack Printing Company, Easton, Pa. See particularly chapters 91-93 for a fuller discussion.

The drug of the invention may be administered parenterally, e.g., intravenously, intramuscularly, intravenously, subcutaneously, or interperitonieally. The carrier or excipient or excipient mixture can be a solvent or a dispersive medium containing, for example, various polar or non-polar solvents, suitable mixtures thereof, or oils. As used herein “carrier” or “excipient” means a pharmaceutically acceptable carrier or excipient and includes any and all solvents, dispersive agents or media, coating(s), antimicrobial agents, iso/hypo/hypertonic agents, absorption-modifying agents, and the like. The use of such substances and the agents for pharmaceutically active substances is well known in the art. Except insofar as any conventional media or agent is incompatible with the active ingredient, use in therapeutic compositions is contemplated. Moreover, other or supplementary active ingredients can also be incorporated into the final composition.

Solutions of the compound may be prepared in suitable diluents such as water, ethanol, glycerol, liquid polyethylene glycol(s), various oils, and/or mixtures thereof, and others known to those skilled in the art.

The pharmaceutical forms suitable for injectable use include sterile solutions, dispersions, emulsions, and sterile powders. The final form must be stable under conditions of manufacture and storage. Furthermore, the final pharmaceutical form must be protected against contamination and must, therefore, be able to inhibit the growth of microorganisms such as bacteria or fungi. A single intravenous or intraperitoneal dose can be administered. Alternatively, a slow long term infusion or multiple short term daily infusions may be utilized, typically lasting from 1 to 8 days. Alternate day or dosing once every several days may also be utilized.

Sterile, injectable solutions are prepared by incorporating a compound in the required amount into one or more appropriate solvents to which other ingredients, listed above or known to those skilled in the art, may be added as required. Sterile injectable solutions are prepared by incorporating the compound in the required amount in the appropriate solvent with various other ingredients as required. Sterilizing procedures, such as filtration, then follow. Typically, dispersions are made by incorporating the compound into a sterile vehicle which also contains the dispersion medium and the required other ingredients as indicated above. In the case of a sterile powder, the preferred methods include vacuum drying or freeze drying to which any required ingredients are added.

In all cases the final form, as noted, must be sterile and must also be able to pass readily through an injection device such as a hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized.

Prevention or inhibition of growth of microorganisms may be achieved through the addition of one or more antimicrobial agents such as chlorobutanol, ascorbic acid, parabens, thermerosal, or the like. It may also be preferable to include agents that alter the tonicity such as sugars or salts.

In a specific embodiment, it may be desirable to administer the compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In another embodiment, the composition can be delivered in a vesicle, in particular a liposome (see Langer, Science 249: 1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the composition can be delivered in a controlled release, or sustained release system. In one embodiment, a pump may be used (see Langer, supra; Sefton, 1987, CRC Crit. Ref. Biomed. Eng. 14: 201; Buchwald et al., 1980, Surgery 88: 507; Saudek et al., 1989, N. Engl. J. Med. 321: 574). In another embodiment, polymeric materials can be used in a controlled release system (see Medical Applications of Controlled Release, Langer and Wise (eds.), CRC Pres., Boca Raton, Fla. (1974); Controlled Drug Bioavailability Drug Product Design and Performance, Smolen and Ball (eds.), Wiley, New York (1984); Ranger and Peppas, J., Macromol. Sci. Rev. Macromol. Chem. 23: 61 (1983); see also Levy et al., Science 228: 190 (1985); During et al., Ann. Neurol. 25: 351 (1989); Howard et al., J. Neurosurg. 71: 105 (1989)). In yet another embodiment, a controlled release system can be placed in proximity of the therapeutic target (e.g., the brain, kidney, stomach, pancreas, and lung), thus requiring only a fraction of the systemic dose (see, e.g., Goodson, in Medical Applications of Controlled Release, supra, vol. 2, pp. 115-138 (1984)). Other controlled release systems are discussed in the review by Langer (1990).

In a specific embodiment where the drug of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88: 1864-1868), etc. Alternatively, a nucleic acid can be introduced intracellularly and incorporated within host cell DNA for expression, by homologous recombination.

### IV. EXAMPLES

The following examples are provided as a guide for a practitioner of ordinary skill in the art. The examples should not be construed as limiting the invention, as the examples merely provide specific methodology useful in understanding and practicing an embodiment of the invention.

### A. Example 1

### Monocyte/T-cell Recruitment

Human PBLs are isolated from the citrate-anticoagulated whole blood of healthy donors or patients with rheumatoid arthritis by dextran sedimentation and density separation over Ficoll-Hypaque. The mononuclear cells are washed and further purified on nylon wool and by plastic adherence, as previously described (Carr 1996). The resulting PBLs (>90% CD3+ T lymphocytes) are cultured in LPS-free RPMI/10% FCS for 15-18 h before use. Memory and naive CD4⁺ T lymphocyte subsets (CD45RO⁺ and CD45RA⁺, respectively) are isolated by negative selection using magnetic cell separation (MACS, Miltenyi Biotec, Bergisch Gladbach, Germany), following the manufacturer's instructions. HUVECs are isolated from umbilical cord veins (jaffe 1973) and established as primary cultures in M199 containing 10% FCS, 8% pooled human serum, 50 μg/ml endothelial cell growth factor (Sigma-Aldrich), 10 U/ml porcine intestinal heparin (Sigma-Aldrich), and antibiotics. Experiments are done on cells at passage two cultured on hydrated Type I collagen gels (Muller 1989) in 96-well culture plates. In certain experiments TNF-α or IL-1β (10 ng/ml and 10 U/ml final concentrations, respectively) or diluted synovial fluid from healthy donors or patients with rheumatoid arthritis are added to the culture media for the final 4-24 h.

The migration of monocytes or T-cells through a layer of endothelial cells is measured. The details of this assay are described in Muller et al., J Exp Med 176: 819-828 (1992) and Muller et al., J Exp Med 178: 449-460 (1993). Transendothelial migration is quantitated by Namarski optics as described in Liao et al., J Exp Med 182: 1337-1343 (1995) and Muller et al., J Exp Med 178: 449-460 (1993). Leukocytes are added to confluent monolayers of HUVECs grown on hydrated collagen gels. After incubation (1 h), nonadherent cells are removed by washing and the remaining adherent and transmigrated cells are fixed in place on the endothelial monolayer by overnight incubation in 2.5% glutaraldehyde in 0.1 M sodium cacodylate buffer at pH 7.4. Multiple high-power fields are observed under microscope and scored. Transmigration data are expressed as the percentage of the total cells that remained with the monolayer that were below the endothelium.

Transendothelial migration is also quantitated on cross-sections of paraffin-embedded monolayers. These specimens are prepared by carefully removing replicate sample monolayers and placing the endothelial surfaces against each other with the collagen gel sides facing outward. This avoids mechanical dislodgement of cells during the embedding process. After substitution in wax, the specimens are bisected so that cuts through the specimen produce cross sections of four monolayer samples (two different portions of each of the two monolayers). Quantitation is performed on three levels of such specimens separated y at least 50 μm so that different areas of the specimen are sampled and the same cells are not counted twice.

### B. Example 2

### Apoptosis Activation and Annexin V Assay

Isolated rheumatoid arthritis synovial fluid MNC and macrophages are incubated with 1 μg/ml of anti-Fas antibody (clone CH11; Beckman Coulter) or irrelevant IgM monoclonal antibody control for 24 hours. Cells are washed twice with cold PBS and then resuspended in 10 mM HEPES, pH 7.4; 140 mM NaCl; 2.5 mM CaCl₂ at a concentration of ˜1×10⁶ cells/ml. 100 μl of the solution (˜1×10 5 cells) is transferred to a 5 ml culture tube. 5 μl of 2.5 μg Annexin V-phycoerythrin and 2.5 μg vital dye 7-AAD are added to each tube, gently mixed and incubated at room temperature in the dark for 15 minutes. 400 μl phosphate buffered saline (PBS) is added to each tube and the cells are analyzed by cell cytometry as soon as possible (within one hour). The percentage of apoptotic cells is measured by the percentage of Annexin V positive cells.

### C. Example 3

### TUNEL Assay

Apoptosis is induced in synovial MNC and macrophages by incubating the cells for 24 h with recombinant TNF (10 ng/ml), or 1 μg/ml anti-Fas, anti-TNF-R1 or anti-TRAIL receptors antibodies 1-2×10⁶ monocytes are centrifuged at 400×G for minutes, the supernatant is discarded and the cells are resuspended in 0.5 ml phosphate buffered saline (PBS). The cells are fixed by adding the cell suspension to 5 ml of 1% (w/v) paraformaldehyde in PBS, placing it on ice for 15 min, washing the cells twice in PBS twice, and finally combining the cells suspended in 0.5 ml PBS with 5 ml ice-cold 70% (v/v) ethanol. The cells stand for a minimum of 30 minutes on ice or in the freezer before proceeding to the staining step.

The tubes are swirled to resuspend the cells and 1.0 ml aliquots of the cell suspensions (˜2-4×10⁵ cells/ml) are removed and placed in 12×75 mm centrifuge tubes. The cell suspensions are centrifuged for 5 min at 300×g and the 70% (v/v) ethanol removed by aspiration. The cells are washed twice by centrifugation and resuspension in PBS plus 0.05% sodium azide, pelleted and then resuspended in 50 μl Staining Solution (TdT enzyme/FITC-dUTP in cacodylate buffered saline). The cells are incubated at 37° C. for at least one hour. The staining is stopped by the addition of 1.0 ml PBS pus 0.05% sodium azide. The cells are pelleted by centrifugation at 300×g for 5 min, resuspended in PBS pus 0.05% sodium azide, and the repelleted. The supernatant is removed by aspiration and the pellet is incubated for 30 minutes at room temperature in the dark. The cells are analyzed by flow cytometry.

### D. Example 4

### Propidium Iodide Staining

Nine-day adherent synovial fluid macrophages are incubated with anti-Fas antibody or control IgM in the presence and absence of the test compound for 24 hours. Cultures are then harvested by 0.02% EDTA, fixing overnight in 70% ethanol, stained with propidium iodide (Roche Molecular Biochemicals, Indianapolis, Ind.), and the subdiploid peak, immediately next to the G₀/G₁ peak (2N), is determined by flow cytometry. It may be necessary to exclude objects with minimal light scatter, possibly representing debris, which would artificially increase the estimate of the subdiploid population. Typically, the percentage of apoptotic synovial macrophages (subdiploid population) increase from 2-5% in absence of an HIF-1α antagonist to 35-40% when HIF-1α activity is completely suppressed.

### E. Example 5

### Anti-Histone Sandwich Assay

Apoptosis is induced by incubating 10⁴ synovial MNC or macrophages with 1 μg/ml anti-Fas antibody (CH11) or TNF-α (10 ng/ml) for 24 h. After the incubation, the cells are pelleted by centrifugation and the supernatant (containing DNA from necrotic cells that leaked through the membrane during incubation) is discarded. The cells are resuspended in Lysis Buffer and incubated 30 min at room temperature. After lysis, cell nuclei and unfragmented DNA are pelleted by centrifugation at 20 000×g for 10 min.

An aliquot of the supernatant (i.e., cytoplasmic fraction) is transferred to anti-histone antibody well of a microtiter plate. The complexes are bound to the plate via streptavidin-biotin interaction. The immobilized antibody-DNA-antibody complexes are washed three times to remove any components that are not immunoreactive. The bound complexes are detected with anti-DNA (peroxidase-conjugated) monoclonal antibodies revealed by a peroxidase substrate and amount of colored product (and thus, of immobilized antibody-histone complexes) is determined spectrophotometrically. The quantitative colorimetric measurement of the DNA-histone complex is proportional to the total amount of apoptotic cells present in the cell population tested.

### F. Example 6

### Western Blot Analysis of HIF-1α Expression

Whole-cell extracts are prepared from synovial MNC and macrophages by lysis in 0.1% NP-40 lysis buffer. 25 to 50% 1 g of extract are analyzed by SDS-PAGE on 12.5% polyacrylamide gels, and transferred to ImmobilonP (Millipore) by semidry blotting. Filters are blocked for 1 h at room temperature in PBS/0.2% Tween-20/5% nonfat dry milk. Filters are blotted with rabbit anti-HIF-1α antibodies at 4° C. in PBS/0.2% Tween-20/2% nonfat dry milk. Filters are washed in PBS/0.2% Tween 20/2% nonfat dry milk and incubated with donkey anti-rabbit or anti-mouse secondary antibody (1:2,000 dilution) conjugated to horseradish peroxidase (Amersham PharmaciaBiotech). Visualization of the immunocomplex is performed using Enhanced Chemiluminescence Plus kit (AmershamPharmacia Biotech).

### G. Example 7

### Capillary Formation Assay

A gel of basement proteins is applied to the bottoms of a 96-well tissue culture plate utilizing the ECMatrix™ system (available from Chemicon Intl., Temecula, Calif.) according to the manufacturer's instructions. HUVECs are isolated from umbilical cord veins and established as primary cultures in M199 containing 10% FCS, 8% pooled human serum, 50 μg/ml endothelial cell growth factor (Sigma-Aldrich), 10 U/ml porcine intestinal heparin (Sigma-Aldrich), and antibiotics. Experiments are done on cells at passage two cultured on hydrated Type I collagen gels (Muller 1989) in 96-well culture plates. The HUVEC cells are resuspended in growth media supplemented with 5% fetal calf serum. 5×10³ cells are applied onto the surface of the polymerized ECMatrix. The cells are incubated overnight in the presence or absence of the test compound. The progression of angiogenesis is quantified by counting the capillary tube branch points formed after the overnight incubation. At least three separate view-fields are counted and the experimental value is determined as the average of the counts associated with the separate view-fields.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A method of alleviating at least one symptom of rheumatoid arthritis comprising administering a therapeutically effective amount of an antagonist of HIF-1α activity to a patient having rheumatoid arthritis.

2. The method of claim 1, wherein the antagonist of HIF-1α activity is a protein.

3. The method of claim 1, wherein the antagonist of HIF-1α activity is a nucleic acid.

4. The method of claim 3, wherein the nucleic acid is an antisense inhibitor.

5. The method of claim 1, wherein the antagonist of HIF-1α activity is a small molecule.

6. The method of claim 1, wherein the antagonist decreases HIF-1α activity by at least 45%.

7. The method of claim 6, wherein the antagonist decreases HIF-1α activity by at least 85%.

8. The method of claim 7, wherein the antagonist decreases HIF-1α activity by at least 95%.

9. The method of claim 1, wherein the patient is a methotrexate resistant patient.

10. The method of claim 1, wherein the patient is a TNF-α blockade nonresponder.

11. A method of decreasing density of synovial cells in a joint comprising administering a therapeutically effective amount of an antagonist of HIF-1α activity to a patient having a condition associated with abnormally increased synovial cell density.

12-20. (canceled)

21. A method of decreasing cartilage degradation in a joint comprising administering a therapeutically-effective amount of an antagonist of HIF-1α activity to a patient having a condition associated with an abnormally high rate of cartilage degradation.

22-30. (canceled)

31. A method of decreasing IL-6 concentration in synovial tissue comprising administering a therapeutically effective amount of an antagonist of HIF-1α activity to a patient having a condition associated with an abnormally high concentration of IL-6 in synovial tissue.

32-40. (canceled)

41. A method of manufacturing a drug for use in the treatment of rheumatoid arthritis comprising:
(a) identifying a compound as useful in the treatment of rheumatoid arthritis by:
(i) comparing an amount of HIF-1α activity in the presence of the compound with an amount HIF-1α activity in the absence of the compound; and
(ii) identifying the compound as useful in the treatment of rheumatoid arthritis when the amount of HIF-1α activity in the presence of the compound is lower than the amount of HIF-1α activity in the absence of the compound; and
(b) formulating said compound for human consumption.

42-80. (canceled)

81. A method identifying a compound useful in the treatment of rheumatoid arthritis, which method comprises:
(a) comparing an amount of HIF-1α activity in the presence of the compound with an amount HIF-1α activity in the absence of the compound; and
(b) selecting the compound as useful in the treatment of rheumatoid arthritis when the amount of HIF-1α activity in the presence of the compound is lower than the amount of HIF-1α activity in the absence of the compound.

82. The method of claim 81 for screening a collection of compounds, further comprising repeating steps (a) and (b) for each compound of the collection, wherein at least one compound of the collection is selected as useful for the treatment of rheumatoid arthritis.

83. The method of claim 81, wherein the compound is selected as useful in the treatment of rheumatoid arthritis when the amount of HIF-1α activity in the presence of the compound is at least 45% lower than the amount of HIF-1α activity in the absence of the compound.

84. The method of claim 83, wherein the compound is selected as useful in the treatment of rheumatoid arthritis when the amount of HIF-1α activity in the presence of the compound is at least 85% lower than the amount of HIF-1α activity in the absence of the compound.

85. The method of claim 84, wherein the compound is selected as useful in the treatment of rheumatoid arthritis when the amount of HIF-1α activity in the presence of the compound is at least 95% lower than the amount of HIF-1α activity in the absence of the compound.

86. The method of claim 81, wherein the amount of HIF-1α activity is measured under hypoxic conditions.

87. The method of claim 81, wherein the amount of HIF-1α activity is measured by an amount of HIF-1α bound to a hypoxia-responsive element (HRE).

88. (canceled)

89. The method of claim 81, wherein the amount of HIF-1α activity is measured by an amount of transcription from an HIF-1α-responsive gene.

90-93. (canceled)

94. The method of claim 81, wherein the amount of HIF-1α activity is measured by a process comprising the step of:
(a) comparing an amount of leukocytes that migrate through at least one layer of endothelial cells in the presence of the compound with an amount of leukocytes that migrate through at least one layer of endothelial cells in the absence of the compound; and
wherein the amount of leukocytes that migrate represents the amount HIF-1α activity.

95-102. (canceled)

103. The method of claim 81, wherein a decrease in HIF-1α activity in the presence of the compound is identified by observing an amount of leukocyte apoptosis in the presence of the compound that is higher than an amount of leukocyte apoptosis in the absence of the compound.

104. The method of claim 103, wherein the leukocytes are macrophages.

105-107. (canceled)

108. The method of claim 104, wherein the amount of macrophage apoptosis is measured by a process comprising the steps of:
(1) exposing a population of cells to an inducer of apoptosis in the presence or absence of the compound; and
(2) measuring the percentage of cells in the population having DNA fragmentation wherein the percentage of cells having DNA fragmentation represents the amount of macrophage apoptosis.

109-112. (canceled)

113. The method of claim 104, wherein the amount of macrophage apoptosis is measured by a process comprising the steps of:
(1) exposing a population of cells to an inducer of apoptosis in the presence or absence of the compound; and
(2) measuring a percentage of cells in the population expressing phosphatidylserine on the extracellular surface of the cell membrane
wherein the percentage of cells expressing phosphatidylserine on the extracellular surface of the cell membrane represents the amount of macrophage apoptosis.

114-117. (canceled)

118. The method of claim 81, wherein the amount of HIF-1α activity is measured by a process comprising the step of:
(a) comparing an amount of angiogenesis in the presence of the compound with an amount of angiogenesis in the absence of the compound;
wherein the amount of angiogenesis represents the amount HIF-1α activity.

119-123. (canceled)

124. A method of alleviating at least one symptom of rheumatoid arthritis, comprising administering an antagonist of HIF-1α activity and an anti-rheumatic drug to a patient having rheumatoid arthritis.

125. The method of claim 124, wherein the anti-rheumatic drug is a symptom-relieving anti-rheumatic drug.

126. The method of claim 124, wherein the anti-rheumatic drug is a disease-modifying anti-rheumatic drug.

127. The method of claim 124, wherein the anti-rheumatic drug is selected from the group of methotrexate, a TNF-α antagonist, an interleukin-1 receptor antagonist and a steroid.

128. The method of claim 124, wherein the patient is a methotrexate resistant patient and the anti-rheumatic drug is methotrexate, a TNF-α antagonist, an interleukin-1 receptor antagonist or a steroid.

129. The method of claim 124, wherein the patient is a TNF-α blockade resistant patient and the anti-rheumatic drug is a TNF-α antagonist, an interleukin-1 receptor antagonist or a steroid.

130. The method of claim 129, wherein the patient is a TNF-α blockade hyperplasia nonresponder.
